SEQUENCE LISTING (8 sequences)

SEQ ID NO: 1 - DNA, 122 bp, synthetic construct
  misc_feature 1..122: note = Synthetic
  source 1..122: mol_type = other DNA
gctccaggcggtgcttgttcctcagcctcttctccttcctgatcgtggcaggcgccaccacgctcttctgcctgctgcactttggagtgatcggcccccagagggaagaggtgagtgcctgg

SEQ ID NO: 2 - DNA, 122 bp, synthetic construct
  misc_feature 1..122: note = synthetic
  source 1..122: mol_type = other DNA
ctccttcctgatcgtggcaggcgccaccacgctcttctgcctgctgcactttggagtgatcggcccccagagggaagaggtgagtgcctggccagccttcatccactctcccacccaagggg

SEQ ID NO: 3 - DNA, 121 bp, synthetic construct
  misc_feature 1..121: note = Synthetic
  source 1..121: mol_type = other DNA
ctggaaaggacaccatgagcactgaaagcatgatccgggacgtggagctggccgaggagcgctccccaagaagacaggggggccccagggctccaggcggtgcttgttcctcagcctcttc

SEQ ID NO: 4 - DNA, 122 bp, synthetic construct
  misc_feature 1..122: note = Synthetic
  source 1..122: mol_type = other DNA
ctagccggccgccctccaccggcgcctccggacgcaacctcgccctggcagggcggtctgcgcgtccagctgattggtggctctgtccagccgccgcacggagctaaaagtgttcttctcca

SEQ ID NO: 5 - DNA, 122 bp, synthetic construct
  misc_feature 1..122: note = Synthetic
  source 1..122: mol_type = other DNA
ccacgtgcgagaaccaagctctgctcctcaagtgacgggggctctgctctgccaggtgaccgcgcaccatttctcgtgcctggcaagctggtccccttctgggtccgggaccaccacgtccc

SEQ ID NO: 6 - DNA, 122 bp, synthetic construct
  misc_feature 1..122: note = Synthetic
  source 1..122: mol_type = other DNA
tgccttccttccttcctctagttcactgcatgtcagccataaatgagtctgcattattaacgtgatgcttttctcatggtctgtgctcatcaatttctcatttccactgacacttctcctca

SEQ ID NO: 7 - DNA, 122 bp, synthetic construct
  misc_feature 1..122: note = Synthetic
  source 1..122: mol_type = other DNA
ctcacccttccctcttggcggtgttgcgggaagggaggagcaactcttcctcaacatcctcgaagctcagtgtctggcattgtaaggccagtggttttcaaacctctgggctcagaaccttt

SEQ ID NO: 8 - DNA, 122 bp, synthetic construct
  misc_feature 1..122: note = Synthetic
  source 1..122: mol_type = other DNA
ggccccacagtgtcacattgacacatggtcagaaatcccacagcatagaggaagtccccccgacttgccactctggttttctgcatttgtgaaacaatggctctctgctctggtggggaaaa

(19) United States
(12) Patent Application Publication
(10) Pub. No.: US 2026/0152795 A1
(43) Pub. Date: Jun. 4, 2026

(54) SYSTEMS AND METHODS FOR DETECTION OF DELIRIUM RISK USING EPIGENETIC MARKERS

(71) Applicant: Gen Shinozaki, Iowa City, IA (US)

(72) Inventor: Gen Shinozaki, Iowa City, IA (US)

(21) Appl. No.: 19/278,453

(22) Filed: Jul. 23, 2025

Related U.S. Application Data
(63) Continuation of application No. 17/276,043, now abandoned.
(60) Provisional application No. 62/731,599, filed on Sep. 14, 2018.

(51) Int. Cl. C12Q 1/6883; G16B 20/20

(52) U.S. Cl. CPC C12Q 1/6883; G16B 20/20; C12Q 2600/154

(57) ABSTRACT

Disclosed is a method of determining a subject's susceptibility to by collecting a biological sample from the subject; determining the methylation status of at least one CpG sequence in the biological sample from the subject; and comparing the methylation status in the biological sample to an established threshold and determining whether or not the subject is likely to suffer delirium. In certain aspects, the at least one CpG sequence is located in a gene selected from TNF-alpha, BNDF, GDNF, LDLRAD4, DAPK1, and IRF8

### CROSS-REFERENCE TO RELATED APPLICATION(S)

This application is a continuation application of U.S. patent application Ser. No. 17/276,043, filed Mar. 12, 2021, entitled “SYSTEMS AND METHODS FOR DETECTION OF DELIRIUM RISK USING EPIGENETIC MARKERS,” which claims priority to International PCT Application No. PCT/US2019/051276 filed on Sep. 16, 2019, which claims priority to U.S. Provisional Application 62/731,599 filed Sep. 14, 2018, and entitled “Systems and Methods for Detection of Delirium Risk Using Epigenetic Markers,” which is hereby incorporated by reference in its entirety under 35 U.S.C. § 119(e).

### SEQUENCE LISTING

A Sequence Listing is submitted herewith in XML format under the file name “9011856_199402.xml” created on Feb. 18, 2026, and having a size of 11,909 Bytes. The Sequence Listing is hereby incorporated by reference in its entirety.

### TECHNICAL FIELD

The disclosure relates to devices and methods for detecting the risk of delirium of patients using epigenetic biomarker data.

### BACKGROUND

The disclosure relates to devices and methods for detecting the risk of delirium in patients utilizing epigenetic biomarkers.

Delirium in elderly patients is common and dangerous. Major risk factors include aging and exogenous insults, such as infection or surgery. In animal models, aging enhances pro-inflammatory cytokine release from microglia in response to exogenous insults. The epigenetic mechanism DNA methylation (DNAm) regulates gene expression and changes with age. Older individuals may have methylation changes that influence the increased cytokine upon insult, but the degree to which aging affects DNAm of cytokine genes is not fully understood.

There is a need in the art for cost-effective and simple methods and devices for accurately predicting and detecting the risk of delirium in patients, particularly elderly patients following insult such as infection or surgery. The disclosed invention provides the physician with the information to make early intervention decisions. The earlier delirium, or a high likelihood of delirium, can be detected, the lower the cost to society and there is also a decreased amount of care required by the patient. The large decrease in hospital stay time this invention will create positively impacts patients, physicians, and any of the healthcare payers.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF SUMMARY

Disclosed herein are various devices and methods for detecting the risk and/or susceptibility of subjects to delirium.

In certain implementations, a method of determining a subject's susceptibility to delirium is disclosed, including the steps of collecting a biological sample from the subject: determining the methylation status of at least one CpG sequence in the biological sample from the subject; and comparing the methylation status in the biological sample to an established threshold and determining whether or not the subject is likely to suffer delirium.

In certain aspects, the at least one CpG sequence is located in a gene selected from TNF-alpha, BNDF, GDNF, LDLRAD4, DAPK1, and IRF8. In further aspects, the at least one CpG sequence includes cg05733135 of chromosome 11 within the BDNF gene and where the detection of methylation indicates the subject is susceptible to delirium. In yet further aspects, the at least one CpG sequence includes cg02328239 of chromosome 5 within the GDNF gene and where the detection of methylation indicates likelihood of experiencing delirium. In still further aspects, the at least one CpG sequence is selected from cg26729380, cg10650821, and cg04425624 of chromosome 6 within the TNF-alpha gene and the detection of demethylation indicates increased likelihood of experiencing delirium. In yet further aspects, the at least one CpG sequence includes cg21295729 of chromosome 18 within the LDLRAD4 gene and the detection of methylation indicates increased likelihood of experiencing delirium. The method where the at least one CpG sequence includes cg10518911 of chromosome 9 within the DAPK1 gene and where the detection of methylation indicates likelihood of experiencing delirium. The method where the at least one CpG sequence includes cg4015794 of chromosome 16 within the IRF8 gene and where the detection of methylation indicates likelihood of experiencing delirium. The method further including determining the ratio of methylated one or more CpG sequences to unmethylated one or more CpG sequences. Implementations of the described techniques may include hardware, a method or process, or computer software on a computer-accessible medium.

Further disclosed herein is a kit for determining a subject's susceptibility to delirium by determining the methylation status of at least one CpG sequence of the subject, the kit including: at least one first nucleic acid primer at least 8 nucleotides in length that is complementary to a bisulfite-converted nucleic acid sequence including a CpG sequence at cg05733135 of chromosome 11 within the BDNF gene, where the at least one first nucleic acid primer detects the methylated CpG sequence.

In certain aspects, the kit further includes at least one second nucleic acid primer at least 8 nucleotides in length that is complementary to a bisulfite-converted nucleic acid sequence including a CpG sequence at cg05733135 of chromosome 11 within the BDNF gene, where the at least one second nucleic acid primer detects the unmethylated CpG sequence. In further aspects, the at least one first nucleic acid primer includes one or more synthetic or non-natural nucleotides. In still further aspects, the kit further includes a solid substrate to which the at least one first nucleic acid primer is bound. In yet further aspects, the substrate is a polymer, glass, semiconductor, paper, metal, gel or hydrogel. In even further aspects, the solid substrate is a microarray or microfluidics card. According to further aspects, the kit further includes a detectable label. In yet to further aspects, the kit further includes at least one second nucleic acid primer at least 8 nucleotides in length that is complementary to a bisulfite-converted nucleic acid sequence including a CpG sequence at cg21295729 of chromosome 18 within the LDLRAD4 gene and where the detection of methylation indicates increased susceptibility to delirium. In yet further aspects, at least one second nucleic acid primer at least 8 nucleotides in length that is complementary to a bisulfite-converted nucleic acid sequence including a CpG sequence selected from cg26729380, cg10650821, and cg4425624 of chromosome 6 within the TNF-alpha gene and the detection of methylation indicates decreased susceptibility to delirium. In even further aspects, the kit includes at least one second nucleic acid primer at least 8 nucleotides in length that is complementary to a bisulfite-converted nucleic acid sequence including a CpG sequence at cg10518911 of chromosome 9 within the DAPK1 gene and the detection of methylation indicates increased susceptibility to delirium.

Further disclosed herein is a computer implemented method for determining whether or not an subject will experience delirium by obtaining self-report data for a user; performing one or more predictive calculations to determine a predicted multisite CpG sequence methylation ratio, a predicted threshold methylation status and predicted delirium of the user; providing a measured multisite CpG sequence unmethylated level and a measured multisite CpG sequence methylation status for the user; generating a predictive score based on the self-report data, the one or more predictive calculations, the measured multisite CpG sequence methylation and unmethylated level and the measured multisite CpG sequence methylation ratio; and outputting a predicted level of delirium based on the predictive score. Other implementations include corresponding computer systems, apparatus, and computer programs recorded on one or more computer storage devices, each configured to perform the actions of the methods.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a correlation table depicting one of the top two CpG sites in TNF-alpha gene, cgcg10650821, that illustrates the relationship between age and DNA methylation from blood samples.

FIG. 2 is a correlation table depicting one of the top two CpG sites in TNF-alpha gene, cgcg01569083, that illustrates the relationship between age and DNA methylation from blood samples.

FIG. 3 is a correlation table that illustrates the relationship between age and TNF-alpha expression in blood.

FIG. 4 is a colored table that depicts the correlation between age and DNA methylation in TNF-alpha, IL-1beta, IL-6, and IL-8 genes.

FIG. 5A is a correlation table for age and blood DNA methylation at cg01360627 in the TNF-alpha gene for delirium cases and non-delirium cases.

FIG. 5B is a correlation table for age and blood DNA methylation at cg23384708 in the TNF-alpha gene for delirium cases and non-delirium cases.

FIG. 5C is a correlation table for age and blood DNA methylation at cg20477259 in the TNF-alpha gene for delirium cases and non-delirium cases.

FIG. 5D is a correlation table for age and blood DNA methylation at cg06825478 in the TNF-alpha gene for delirium cases and non-delirium cases.

FIG. 6 is a colored table depicting the correlation between age and blood DNA methylation at 24 CpG's in the TNF-alpha gene compared between delirium cases and non-delirium cases.

FIG. 7 is a colored table that shows the correlation of age and DNA methylation at 24 CpG's in the TNF-alpha gene among glial cells, neuron samples, and blood samples from six patients.

FIG. 8A shows data showing DNAm change with age, changes in brain IL-6 cg23731304 DNAm and age correlated significantly.

FIG. 8B shows data showing DNAm change with age, changes in blood IL-6 cg23731304 DNAm showed a similar trend as brain with aging.

FIG. 8C showing data showing DNAm change with age, changes in blood and brain IL-6 cg23731304 correlated significantly.

FIG. 9 shows correlations of age and DNAm levels from blood samples obtained from the GTP cohort.

FIG. 10 shows correlations of age and TNFalpha expression level from blood samples obtained from the GTP cohort.

FIG. 11 shows demographic data of delirium cases and non-delirium controls.

FIG. 12 shows correlation of age and blood DNAm at 24 CpGs in the TNF-alpha gene compared between delirium cases vs non-delirium controls.

FIG. 13 is a volcano plot for the distribution of individual CpG differences and their corresponding logarithmic transformed p-values (n=87).

FIG. 14 shows the top 20 differentially methylated CpGs between delirium cases and non-delirium controls.

FIG. 15 shows results of the top 20 pathways of GO analysis with CpGs differentially methylated between delirium cases and non-delirium controls

FIG. 16 shows results of the top 20 pathways of KEGG analysis with CpGs differentially methylated between delirium cases and non-delirium controls.

FIG. 17 shows results of the significant pathways of GO analysis with CpGs differentially methylated between delirium cases and non-delirium controls.

FIG. 18 shows correlation between chronological age and DNAm age in delirium (n=43) vs controls (n=44).

FIG. 19 is a colored table showing correlations between age and DNAm levels of neurotrophic genes in blood samples obtained from the GTP cohort.

FIG. 20A shows correlation between age and beta value in the top 2 CpGs.

FIG. 20B shows correlation between age and beta value in the top 2 CpGs.

FIG. 21 is a colored table showing correlations between age and DNAm levels at 226 CpGs in neurotrophic genes in blood samples obtained from the GTP cohort.

FIG. 22 is a colored tables showing correlations between age and DNAm levels of neurotrophic genes in brain samples obtained from the NSG cohort.

FIG. 23 is a colored table showing correlations between age and DNAm levels at 201 CpGs in neurotrophic genes in brain samples obtained from the NSG cohort.

FIG. 24 is a colored table showing correlation of age and blood DNAm at 83 CpGs in the BDNF gene compared between delirium cases vs non-delirium controls in the EOD cohort.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a patient, including a mammal (e.g., a human), and includes: (i) preventing the disease from occurring in a patient that can be predisposed to the disease but has not yet been diagnosed as having it; (ii) inhibiting the disease, i.e., arresting its development; or (iii) relieving the disease, i.e., causing regression of the disease. In one aspect, the patient is a mammal such as a primate, and, in a further aspect, the patient is a human.

CpG islands are short stretches of DNA in which the frequency of the CpG sequence is higher than other regions. The “p” in the term CpG indicates that cytosine (“C”) and guanine (“G”) are connected by a phosphodiester bond. CpG islands are often located around promoters of housekeeping genes and many regulated genes. At these locations, the CpG sequence is not methylated in active genes. By contrast, the CpG sequences in inactive genes are usually methylated to suppress their expression.

The methylation of a CpG island can be determined by the those skilled in the art using any method suitable to determine such methylation. For example, the art worker can use a bisulfite reaction-based method for determining such methylation.

Disclosed is a screening kit for determining whether a subject has a predisposition to, or likelihood of having delirium (e.g. susceptibility) including at least one probe specific for a TNF-alpha, LDLRAD4, DAPK1, IRF8 IL-1beta, Il-2, IL-6, and/or IL-8 genes. In certain embodiments, the kit further includes a solid substrate, wherein each probe is bound onto the substrate in a distinct spot. In certain embodiments, the probe detects methylation at CpG residue of BDNF, GDNF, TNF-alpha, LDLRAD4, DAPK1, and/or IRF8. In certain embodiments, the substrate is a polymer, glass, semiconductor, paper, metal, gel or hydrogel. In certain embodiments, the kit further includes at least one control probe, wherein the at least one control probe is bound onto the substrate in a distinct spot. In certain embodiments, the solid substrate is a microarray or microfluidics card. In certain embodiments, the probe is an oligonucleotide probe or a nucleic acid derivative probe.

The present disclosure provides a diagnostic method using bisulfite treated DNA for determining whether a subject has the likelihood of having or developing delirium by determining methylation status of a CpG dinucleotide sequence in a peripheral blood cell or its derivative, wherein the methylation status of the CpG sequence is associated with delirium. In certain embodiments, the method determines the methylation status of a plurality of CpG dinucleotide sequences. Such a plurality may be any integer between 1 and 10,000, such as at least 100.

Any one or more of the CpG sequences in which methylation status has been associated with susceptibility to delirium can be used in the methods and/or kits herein to determine the predictive value (e.g., representing the likelihood developing delirium). These include, but are not limited to the CpG sequences listed in Table 3. Further included is any other CpG sequence disclosed herein as associated with susceptibility to delirium. It would be understood that, particularly for determining delirium susceptibility, the more CpG sequences (i.e., CpG sequences in which methylation status has been associated with delirium susceptibility) are evaluated, the more accurate the predictive value will be.

TABLE 1

| | | | | | Position | |
|---|---|---|---|---|---|---|
| SEQ | | | | | (Human | |
| ID | | | Forward_Sequence with | Chromo- | Genome | |
| NO. | Gene | Probe | CpG site marked in [ ] | some | build 37) | Strand |
| 1 | TNF- | cg26729380 | GCTCCAGGCGGTGCTTGTTCCTCAG | 6 | 31543655 | F |
| | alpha | | CCTCTTCTCCTTCCTGATCGTGGCA | | | |
| | | | GGCGCCACCA[CG]CTCTTCTGCCT | | | |
| | | | GCTGCACTTTGGAGTGATCGGCCCC | | | |
| | | | CAGAGGGAAGAGGTGAGTGCCTGG | | | |
| 2 | TNF- | cg10650821 | CTCCTTCCTGATCGTGGCAGGCGCC | 6 | 31543686 | F |
| | alpha | | ACCACGCTCTTCTGCCTGCTGCACT | | | |
| | | | TTGGAGTGAT[CG]GCCCCCAGAGG | | | |
| | | | GAAGAGGTGAGTGCCTGGCCAGCCT | | | |
| | | | TCATCCACTCTCCCACCCAAGGGG | | | |
| 3 | TNF- | cg04425624 | CTGGAAAGGACACCATGAGCACTGA | 6 | 31543565 | R |
| | alpha | | AAGCATGATCCGGGACGTGGAGCTG | | | |
| | | | GCCGAGGAGG[CG]CTCCCCAAGAA | | | |
| | | | GACAGGGGGGCCCCAGGGCTCCAGG | | | |
| | | | CGGTGCTTGTTCCTCAGCCTCTTC | | | |
| 4 | BDNF | cg05733135 | CTAGCCGGCCGCCCTCCACCGGCGC | 11 | 27740876 | F |
| | | | CTCCGGACGCAACCTCGCCCTGGCA | | | |
| | | | GGGCGGTCTG[CC]CGTCCAGCTGA | | | |
| | | | TTGGTGGCTCTGTCCAGCCGCCGCA | | | |
| | | | CGGAGCTAAAAGTGTTCTTCTCCA | | | |
| 5 | GDNF | cg02328239 | CCACGTGCGAGAACCAAGCTCTGCT | 5 | 37837463 | R |
| | | | CCTCAAGTGACGGGGGCTCTGCTCT | | | |
| | | | GCCAGGTGAC[CG]CGCACCATTTC | | | |
| | | | TCGTGCCTGGCAAGCTGGTCCCCTT | | | |
| | | | CTGGGTCCGGGACCACCACGTCCC | | | |
| 6 | LDLRAD4 | cg21295729 | TGCCTTCCTTCCTTCCTCTAGTTCA | 18 | 13580042 | R |
| | | | CTGCATGTCAGCCATAAATGAGTCT | | | |
| | | | GCATTATTAA[CG]TGATGCTTTTC | | | |
| | | | TCATGGTCTGTGCTCATCAATTTCT | | | |
| | | | CATTTCCACTGACACTTCTCCTCA | | | |
| 7 | DAPK1 | cg10518911 | CTCACCCTTCCCTCTTGGCGGTGTT | 9 | 90172046 | F |
| | | | GCGGGAAGGGAGGAGCAACTCTTCC | | | |
| | | | TCAACATCCT[CG]AAGCTCAGTGT | | | |
| | | | CTGGCATTGTAAGGCCAGTGGTTTT | | | |
| | | | CAAACCTCTGGGCTCAGAACCTTT | | | |
| 8 | IRFS | cg04015794 | GGCCCCACAGTGTCACATTGACACA | 16 | 85947866 | R |
| | | | TGGTCAGAAATCCCACAGCATAGAG | | | |
| | | | GAAGTCCCCC[CG]ACTTGCCACTC | | | |
| | | | TGGTTTTCTGCATTTGTGAAACAAT | | | |
| | | | GGCTCTCTGCTCTGGTGGGGAAAA | | | |

In certain embodiments, disclosed is a kit for determining whether a subject has a predisposition to, or likelihood of having delirium comprising at least one first nucleic acid primer at least 8 nucleotides in length that is complementary to a bisulfite-converted nucleic acid sequence comprising a CpG sequence chosen from cg26729380, cg10650821, or cg04425624 of chromosome 6 within the TNF-alpha gene, wherein the at least one first nucleic acid primer detects the unmethylated CpG sequence and demethylation is indicative of susceptibility to delirium. In certain further embodiments, the kit further comprises second and/or third primer that a bind upstream or downstream of the CpG sequence at cg26729380, cg10650821, or cg04425624 of chromosome 6 within the TNF-alpha gene.

In certain implementations, the disclosed kit comprises at least one first nucleic acid primer at least 8 nucleotides in length that is complementary to a bisulfite-converted nucleic acid sequence comprising a CpG sequence at a position chosen from U.S. Pat. Nos. 31,543,655, 31,543,686, or 31543565 of chromosome 6 within the TNF-alpha gene, wherein the at least one first nucleic acid primer detects the unmethylated CpG dinucleotide and demethylation is indicative of susceptibility to delirium. In certain further embodiments, the kit further comprises second and/or third primer that a bind upstream or downstream of the CpG dinucleotide at position 31543655, 31543686, or 31543565 of chromosome 6 within the TNF-alpha gene.

In certain embodiments, disclosed is a kit for determining whether a subject has a predisposition to, or likelihood of having delirium comprising at least one first nucleic acid primer at least 8 nucleotides in length that is complementary to a bisulfite-converted nucleic acid sequence comprising a CpG sequence at cg05733135 of chromosome 11 within the BDNF gene, wherein the at least one first nucleic acid primer detects the methylated CpG sequence and methylation is indicative of susceptibility to delirium.

In certain implementations, the disclosed kit comprises at least one first nucleic acid primer at least 8 nucleotides in length that is complementary to a bisulfite-converted nucleic acid sequence comprising a CpG dinucleotide at position 27740876 of chromosome 11 within the BDNF gene, wherein the at least one first nucleic acid primer detects the methylated CpG dinucleotide and methylation is indicative of susceptibility to delirium

In certain embodiments, the disclosed kit for determining whether a subject has a predisposition to, or likelihood of having delirium comprises at least one first nucleic acid primer at least 8 nucleotides in length that is complementary to a bisulfite-converted nucleic acid sequence comprising a CpG sequence at cg02328239 of chromosome 5 within the GDNF gene, wherein the at least one first nucleic acid primer detects the methylated CpG sequence and methylation is indicative of susceptibility to delirium.

In certain implementations, the disclosed kit comprises at least one first nucleic acid primer at least 8 nucleotides in length that is complementary to a bisulfite-converted nucleic acid sequence comprising a CpG dinucleotide at position 37837463 of chromosome 5 within the GDNF gene, wherein the at least one first nucleic acid primer detects the methylated CpG dinucleotide and methylation is indicative of susceptibility to delirium.

In certain embodiments, disclosed is a kit for determining whether a subject has a predisposition to, or likelihood of having delirium comprising at least one first nucleic acid primer at least 8 nucleotides in length that is complementary to a bisulfite-converted nucleic acid sequence comprising a CpG sequence at cg21295729 of chromosome 18 within the LDLRAD4 gene, wherein the at least one first nucleic acid primer detects the methylated CpG sequence and methylation is indicative of susceptibility to delirium.

In certain implementations, the disclosed kit comprises at least one first nucleic acid primer at least 8 nucleotides in length that is complementary to a bisulfite-converted nucleic acid sequence comprising a CpG dinucleotide at position 37837463 of chromosome 18 within the LDLRAD4 gene, wherein the at least one first nucleic acid primer detects the methylated CpG dinucleotide and methylation is indicative of susceptibility to delirium.

In certain embodiments, the disclosed for determining whether a subject has a predisposition to, or likelihood of having delirium comprises at least one first nucleic acid primer at least 8 nucleotides in length that is complementary to a bisulfite-converted nucleic acid sequence comprising a CpG sequence at cg10518911 of chromosome 9 within the DAPK1 gene, wherein the at least one first nucleic acid primer detects the methylated CpG sequence and methylation is indicative of susceptibility to delirium.

In certain implementations, the disclosed kit comprises at least one first nucleic acid primer at least 8 nucleotides in length that is complementary to a bisulfite-converted nucleic acid sequence comprising a CpG dinucleotide at position 90172046 of chromosome 9 within the DAPK1 gene, wherein the at least one first nucleic acid primer detects the methylated CpG dinucleotide and methylation is indicative of susceptibility to delirium.

In certain embodiments, disclosed is a kit for determining whether a subject has a predisposition to, or likelihood of having delirium comprising at least one first nucleic acid primer at least 8 nucleotides in length that is complementary to a bisulfite-converted nucleic acid sequence comprising a CpG sequence at cg04015794 of chromosome 16 within the IRF8 gene, wherein the at least one first nucleic acid primer detects the methylated CpG sequence and methylation is indicative of susceptibility to delirium.

In certain implementations, the disclosed kit comprises at least one first nucleic acid primer at least 8 nucleotides in length that is complementary to a bisulfite-converted nucleic acid sequence comprising a CpG dinucleotide at position 85947866 of chromosome 16 within the IRF8 gene, wherein the at least one first nucleic acid primer detects the methylated CpG dinucleotide and methylation is indicative of susceptibility to delirium.

In certain embodiments, the disclosed methods are implemented by way of a PCR (polymerize chain reaction) assay. In some cases, this will take the form of real time PCR assays (RT-PCR) assays. In certain embodiments of these PCR assays, a kit may contain two primers that specifically amplify a region of a BDNF, GDNF, LDLRAD4, DAPK1, and/or IRF8 locus and gene specific probe that selectively recognizes the amplified region. Together, the primers and the gene specific probes are referred to as a primer-probe set. By measuring the amount of gene specific probe that has hybridized to an amplified segment at a given point of the PCR reaction or throughout the PCR reaction, one who is skilled in the art can infer the amount of nucleic acid originally present at the start of the reaction. In some cases, the amount of probe hybridized is measured through fluorescence spectrophotometry. The number of primer-probe sets can be any integer between 1 and 10,000 probes, such as 1, 2, 3, 4, 5, 6, 7, 8, 9, 10, . . . 9997, 9998, 9999, 10,000. In one kit, all of the probes may be physically located in a single reaction well or in multiple reaction wells. The probes may be in dry or in liquid form. They may be used in a single reaction or in a series of reactions. In certain embodiments, the probe is an oligonucleotide probe. In certain embodiments, the probe is a nucleic acid derivative probe.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

“Naturally occurring” is used to describe a composition that can be found in nature as distinct from being artificially produced. For example, a nucleotide sequence present in an organism, which can be isolated from a source in nature and which has not been intentionally modified by a person in the laboratory, is naturally occurring.

Further disclosed is a computer implemented method for determining whether or not an subject will experience delirium, the method comprising: obtaining self-report data for a user; performing one or more predictive calculations to determine a predicted multisite CpG sequence methylation ratio, a predicted threshold methylation status and predicted delirium of the user; providing a measured multisite CpG sequence unmethylated level and a measured multisite CpG sequence methylation status for the user; generating a predictive score based on the self-report data, the one or more predictive calculations, the measured multisite CpG sequence methylation and unmethylated level and the measured multisite CpG sequence methylation ratio; and outputting a predicted level of delirium based on the predictive score.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

To test whether DNAm patterns among cytokine genes correlate with aging, we first used a dataset based on blood samples from 265 GTP subjects (age range 18-65 years old).

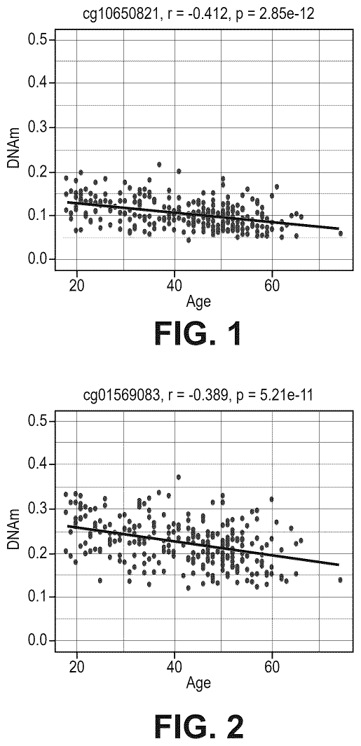

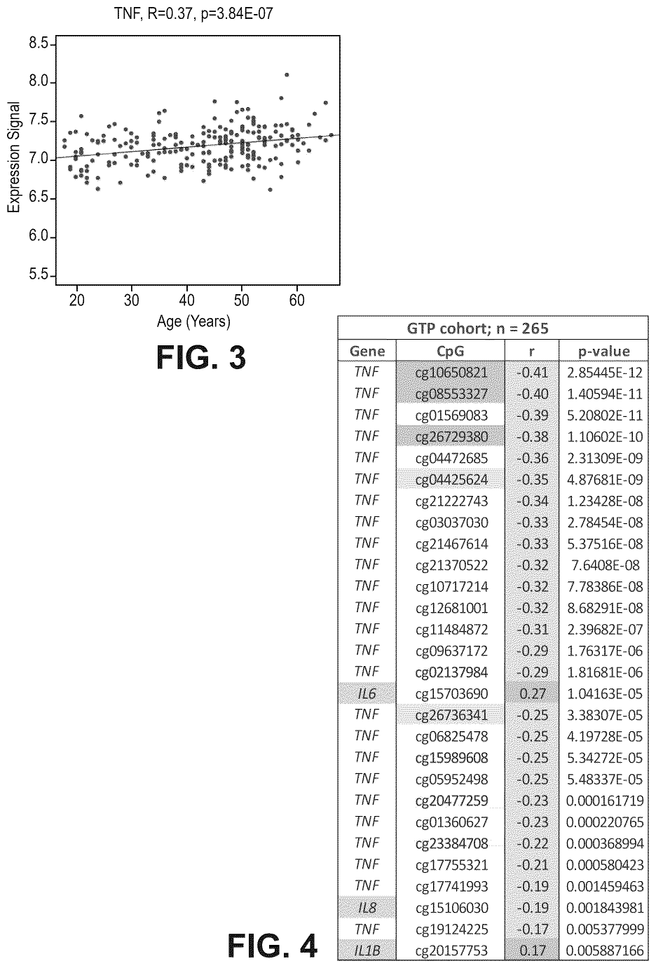

FIG. 1 illustrates cg10650821 site of TNF-alpha which provides evidence of a decrease in DNAm as a patient becomes older with r=−0.41 and p-value of 2.85×E−12. FIG. 2 illustrates a similar result at cg01569083 with r=−0.389 and a p-value of 5.21×E−11. Furthermore, FIG. 3 shows the correlation between age and TNF-alpha expression levels in blood. The result was r=0.37 and a p-value of 3.84×E−7. The correlation tables show and support the claim that DNAm change along with aging and increase expression in cytokine genes can be used as an epigenetic marker for the indication of risk of delirium.

As shown in FIGS. 1-4, the highest correlation was at cg 10650821 (r=−0.41; p-value of 2.85×E−12) (FIGS. 1-4). All 27 CpGs in TNF-alpha were at least nominally significant and 8 of them were significant at genome-wide significance levels (p<5×E−8) (FIG. 3). Moreover, testing of the expression levels of these cytokines confirmed that TNF-alpha expression in blood had a significant positive association with aging (r=0.37, p=3.84×E−7) (FIG. 3).

FIG. 4 provides the rho-value and p-value for 28 CpG's which exhibited an association between age and DNAm levels from blood samples. A majority of the CpG's were from the TNF-alpha gene. The CpG's from other genes are highlighted in grey in the table.

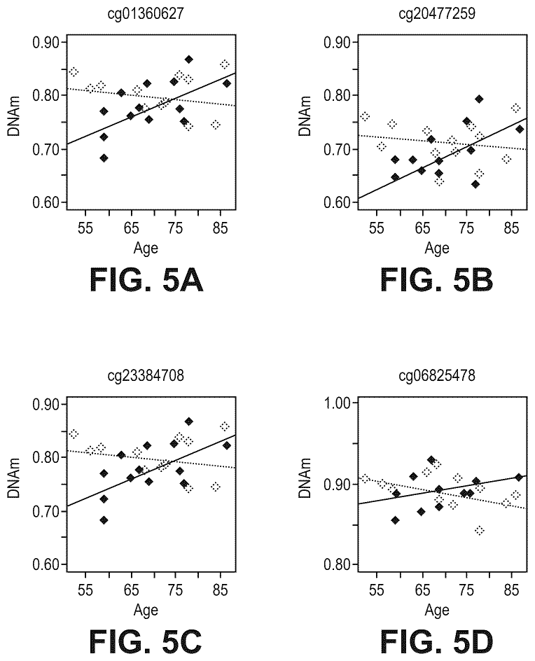

FIG. 5A, FIG. 5B, FIG. 5C, and FIG. 5D provide evidence of the patterns of DNAm over time in patient with and without delirium at TNF-alpha locations cg01360627, cg23384708, cg20477259, cg06825478. In the tables, red indicates patients with delirium while the blue data points indicate patients without delirium. All four show distinct patterns of the patients with delirium and those without delirium at statistically significant value of a=0.05.

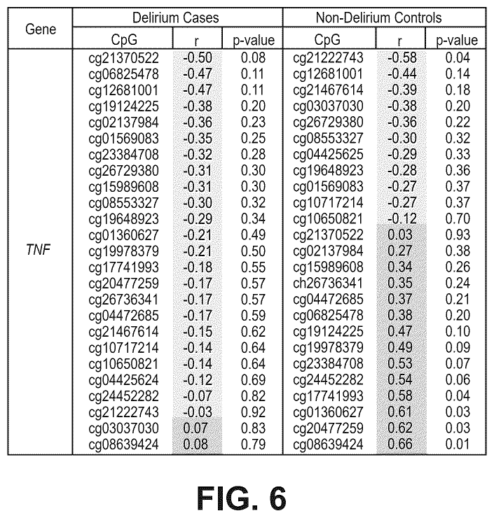

FIG. 6 is a table that displays the same correlations as shown in FIG. 5A, FIG. 5B, FIG. 5C, and FIG. 5D, but for 24 CpG's. The rho-value cells in red indicate a negative correlation between DNAm and age and blue indicates a positive correlation. In the non-delirium patients approximately half of the CpG's have a positive correlation and half have a negative correlation. In the delirium patients all but two of the CpG's tested resulted in a negative correlation between age and DNAm.

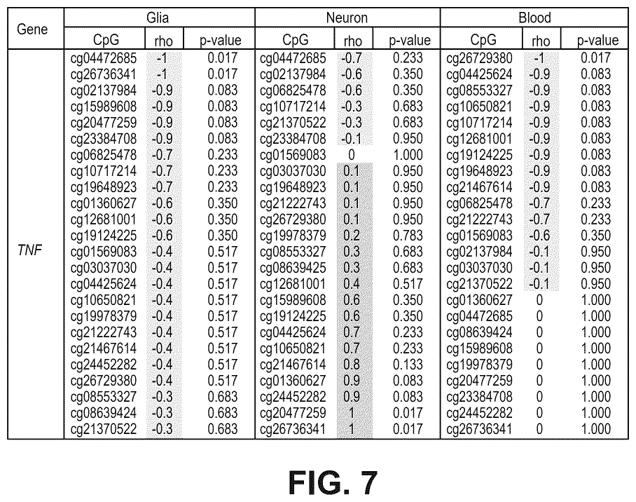

FIG. 7 depicts the correlation of age and DNAm at 24 CpG's of the TNF-alpha gene among glial cell samples, neuron cells, and blood cell samples. As before, red cells indicate a negative correlation between age and DNAm while blue cells indicate a positive correlation. This illustration provides support for the claim that glial cell DNA methylation changes with aging in cytokine genes.

According to certain embodiments, TNF-alpha gene provides the predictive information required for a physician to make an informed decision. After a biological sample, such as a blood sample, is collected and amplified it may be sequenced and analyzed. The analysis covers multiple CpG sites within the TNF-alpha gene. Utilizing multiple CpG sites decreases the possibility for false positives.

In the present Example, we used samples from two different cohorts to examine the association between aging and DNAm of cytokine genes. One cohort was a set of patients with medically intractable epilepsy from whom neurosurgically resected brain tissue and blood samples were collected simultaneously [neurosurgery cohort (NSG)]. Additionally, blood samples with DNAm and expression data were used from an independent, large cohort of 265 subjects from the Grady Trauma Project (GTP) (Smith et al., 2011).

### Methods and Materials

### Study Subjects and Sample Collection for NSG Cohort

A more detailed overview of study participants and sample collection process has been described previously (Braun et al., 2017; Shinozaki et al., 2017). Briefly, 21 subjects with medically intractable epilepsy undergoing neurosurgery were recruited for this study between March 2014 and April 2017 at the University of Iowa Hospitals and Clinics. This study was approved by the University of Iowa's Human Subjects Research Institutional Review Board. Written informed consent was obtained, and whole blood samples were collected in EDTA tubes, saliva with the Oragene DISCOVER™ kit (DNA Genotek Inc., OGR-500), and buccal tissue with swabs (Puritan, 25-1506 1 PF TT MC). Resected brain tissue samples were immediately stored and transported on dry ice, and a portion of each brain region was sent to pathology. All samples were stored at −80° C. Typically, blood samples were taken at the end of surgery in the operating room, and saliva and buccal swabs were collected within 2 days after the operation. FACS was performed as previously described (Braun et al., 2017; Shinozaki et al., 2017).

### Sample Processing and Epigenetics Platform

Methylome assays were performed as previously described. Briefly, genomic DNA from saliva, buccal, and whole blood were isolated with the MasterPure™ DNA extraction kit (Epicenter, MCD85201). DNA was bisulfite-converted using the EZ DNA Methylation™ Kit (Zymo Research, D5002). The Infinium HumanMethylationEPIC BeadChip™ Kit (Illumina, WG-317-1002) was used to analyze DNAm of 21 subjects with brain, blood, saliva, and buccal samples. Raw data was processed using the R packages RnBeads and Minfi (Aryee et al., 2014; Fortin et al., 2017); this enables quality control checks, data filtering, and normalization of the data in addition to differential methylation analyses (Aryee et al., 2014; Assenov et al., 2014; Fortin et al., 2017).

### Statistical Analysis

All statistical analyses were performed in R (Team, 2017). DNAm correlation was calculated with Pearson's correlation using the average methylation for each tissue. The correlation of cytokine genes was performed with CpGs from those genes present on the array. The degree of correlation between aging and DNAm was calculated for each CpG in the cytokine genes tested. The correlation coefficient and its significance level was calculated with Spearman's test using R package limma (Smyth et al., 2005).

DATA from GTP Cohort

Detailed sample collection for the Grady Trauma Project can be found in Smith et al. (2011) (Smith et al., 2011). Illumina HumanMethylation450 DNAm data was processed according to a previously described pipeline (Ratanatharathorn et al., 2017). Briefly, the Infinium protocol was assessed at each step by visual inspection of control probes, after which background normalized beta values, methylated signals, unmethylated signals, and detection p-values were exported to R (Ihaka and Gentleman, 1996). Based on the R package CpGassoc, low-intensity samples (probe detection call rates <90% and an average intensity value less than half the overall median or 2,000 arbitrary units) and proves with detection p-values >0.001 were removed (Barfield et al., 2012). Sex chromosome cross-hybridizing probes were also removed (Chen et al., 2013). Type I and Type II probes were normalized with Beta Mixture Quantile Normalization after which the ComBat procedure was run twice to remove chip then position effects while controlling for gender and PTSD status (Leek et al., 2012; Teschendorff et al., 2013). Prior to analysis, β-values were logit transformed into M-values (Du et al., 2010).

For the gene expression analysis, RNA was extracted from whole blood collected in Tempus tubes at about 8:30 AM for all participants. All samples had Bioanalyzer RNA Integrity Number (RIN) □ 16. Probes were considered sufficiently expressed if they had a detection p-value of <0.01 in 5% of the samples. In total 15,877 probes met these criteria. The array was log 2 transformed and normalized using the Supervised Normalization Method (Mecham et al., 2010).

### Results

### Patient Demographics

### NSG Study

We analyzed brain and peripheral tissue samples from 21 NSG subjects, and among them, seven were female and the average age was 31 years (SD±16.4). The resected and analyzed brain regions included the temporal cortex (11 individuals), the frontal cortex (four individuals), the hippocampus (four individuals), and the occipital area (two individuals). Fluorescence-activated cell sorting (FACS) was performed on brain tissues to separate cells positive for a neuronal marker, resulting in six neuronal-positive (neuron) samples and 13 neuronal-negative (glia) ones with sufficient DNA quantity for analysis.

### GTP Study

Detailed information about the GTP cohort has been described previously (Smith et al., 2011). Briefly, the total sample size with available DNAm and expression data is 265, 71% were female, and the average age was 42 years (SD±12) at the time of collection. Ninety-four percent of samples were received from African American individuals and 5% were received from Caucasian individuals.

DNA Methylation from NSG Brain and Blood Samples

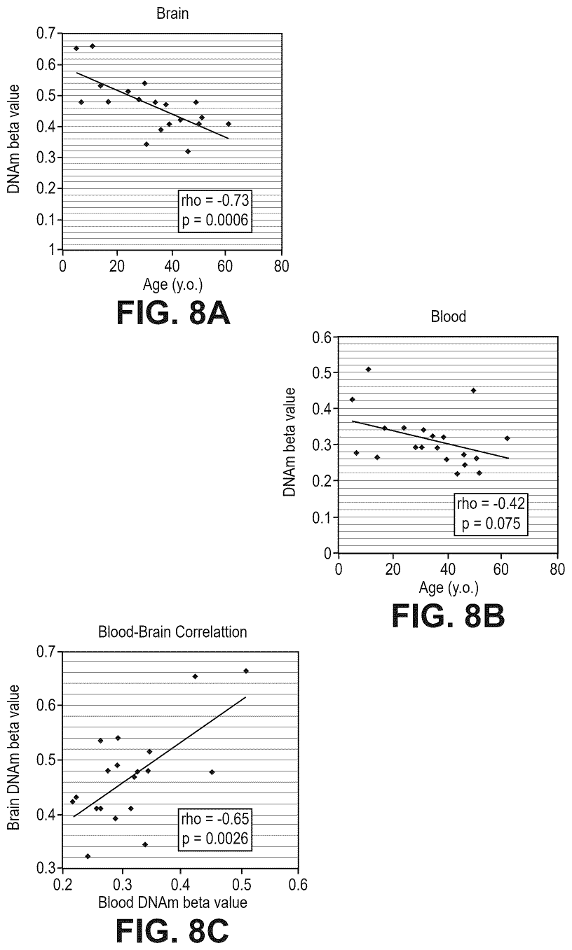

We first used our dataset from the NSG study (Braun et al., 2017; Shinozaki et al., 2017) to see if DNAm patterns among cytokine genes are correlated with aging in the brain. We looked at DNAm levels and their association with age across subjects (age rage 5-62 years) in brain tissue resected during neurosurgery. We chose the pro-inflammatory cytokine genes, IL-1beta, IL-6, and TNF-alpha, to test, which includes a total of 52 CpGs. Six CpGs correlated with aging at nominal significance (p<0.05). Given that one in 20 tests would be significant by chance alone, in 52 CpGs we expected to have 2.6 that are significant by chance. Our top hit was in IL-6 (cg23731304), which had a rho of −0.73 and a p-value of 0.00060 (FIG. 8a). This was significant even after correction for multiple testing at the level of 0.05/52=0.00096. We then looked at this specific CpG and its DNAm trend in blood, and we found that its rho was-0.42 and the p-value was 0.075 (FIG. 8b). The correlation of DNAm between brain and blood at this CpG was also significant (rho=0.65; p-value=0.0026) (FIG. 8c).

DNA methylation and expression from GTP cohort, a total of 265 subjects' blood samples

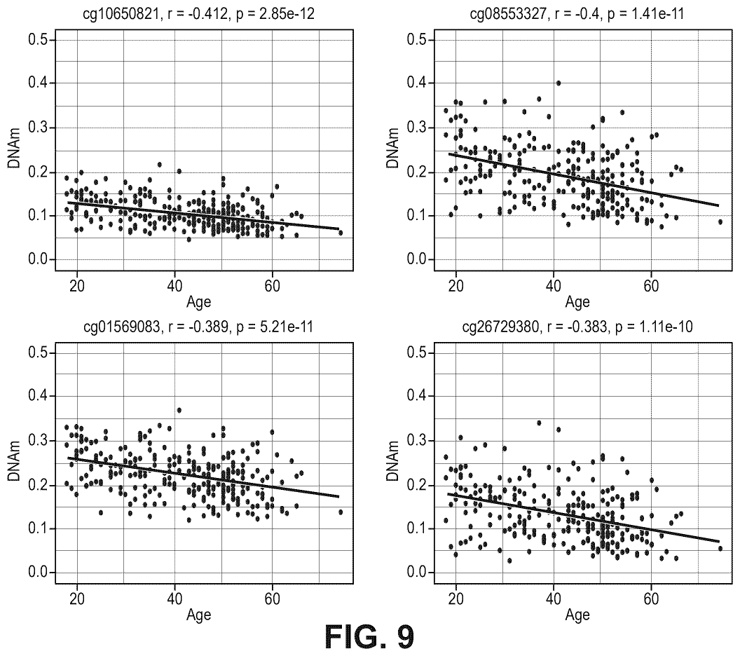

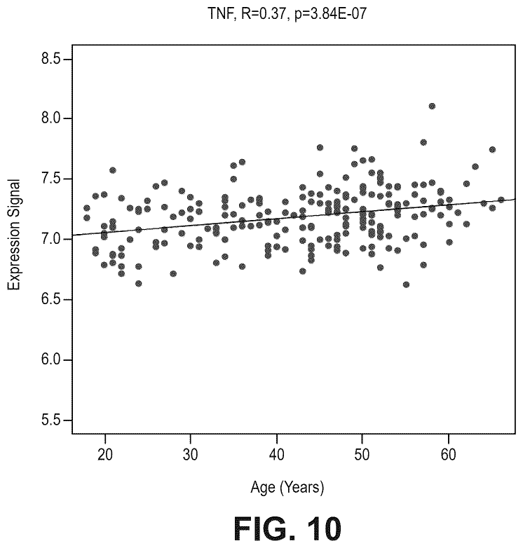

Similar to the NSG preliminary analysis, we sought to investigate the association between DNAm of IL-1beta, IL-6, IL-8, and TNF-alpha and aging among the GTP cohort. The dataset included 74 CpGs, and among them DNAm levels at 14 CpGs was associated with aging at nominal significance (p<0.05). There were 38 CpGs that were significantly associated at nominal levels with aging, predominantly with TNF-alpha represented. In fact, all 27 CpGs in TNF-alpha were at least nominally significant (FIG. 7). Eight CpGs were significant at genome-wide significant levels (p<5×E−8), all in TNF-alpha (FIG. 7). The top hit was at cg10650821 (r=−0.41; p-value of 2.85×E−12) (FIG. 6, FIG. 9). Moreover, when expression levels of these cytokines were tested for a subgroup of 215 subjects with available expression data, TNF-alpha expression was significantly positively associated with aging as expected (r=0.37, p=3.84×E−7) (FIG. 10).

NSG Samples Revisited, DNA Methylation from Blood and FACS-Sorted Brain

We sought to determine if the GTP top findings were also represented in the NSG study. Three of the top four hits (cg10650821, cg08553327, and cg26729380, all from TNF-alpha, highlighted in orange in FIG. 7) were correlated with aging at nominal significance in blood (N=21), resulting in an independent cohort replicating the finding.

Next, we tested if these associations of DNAm in TNF-alpha with aging in the GTP cohort were similarly present in our NSG brain tissues. We used data from just the six subjects who have samples from each tissue type (whole brain, neuronal-positive, neuronal-negative, and blood) to better compare the trend in correlation between age and DNAm at all 24 CpGs tested in the TNF-alpha gene. In both whole brain tissue and FACS-sorted neuronal-positive (neuron) cells, no trends were identified, but in FACS-sorted neuronal-negative (glial) cells nominal significant associations with aging were seen at cg04472685 and cg26736341 (p=0.017, light blue highlight in FIG. 6. FIG. 7), and similar trends were seen in next four top CpG sites (dark blue highlight in FIG. 6, FIG. 7).

All 24 CpGs showed a negative correlation in glia (rho=−1˜−0.3), whole brain (rho=−0.9˜0), and blood (rho=−1˜0), whereas neuronal-positive cells showed variable rho value ranges from negative to positive (rho=−0.7˜1) (FIG. 7). This shows a contrast between glia and neuron cell types in trends of DNAm associating with aging, as well as the similarity in trends between glia and blood. Of note, though, the sample size was very small, which could limit the ability to detect statistically significant trends.

### DISCUSSION

The NSG data from 21 samples showed that in human brain the DNAm level of one CpG at a promotor region of the pro-inflammatory cytokine gene (IL-6) decreases with age, which could potentially associate with an increase in IL-6 expression. Also, the DNAm level of this CpG in IL-6 is correlated between brain and blood, and in blood, the DNAm shows a similar trend with aging. These findings suggest that blood DNAm of this IL-6 CpG has the utility of an epigenetic biomarker of age-related changes in brain. In fact, there is also a report that shows a low DNAm at a single CpG in IL-6 is associated with heightened expression from blood obtained from human subjects (Nile et al., 2008).

The data from an independent cohort in the GTP cohort revealed an age-associated DNAm decrease along with aging at multiple CpGs from blood samples. The pattern is very consistent with the NSG cohort. The top signal showed a genome-wide significant p-value of 2.85×E−12. Adjusting for multiple comparison correction of the 74 CpG sites tested, 28 CpGs were significant at 0.05/74=0.00068, as listed in FIG. 7 (24 CpGs from GTP). The majority of these findings from blood DNAm were in TNF-alpha and showed a decrease with aging, which is consistent with literature (Gowers et al., 2011). Moreover, expression of TNF-alpha was positively correlated with aging as expected based on negative correlation of DNAm with age. On the contrary, expression of other pro-inflammatory cytokines except TNF-alpha was not correlated with aging from this cohort. This could indicate that other cytokines are stabilized in certain range when human body is in stable condition. However, because of underline DNAm change along with aging, when individuals are exposed to exogenous insults, they are more prone to enhanced expression, which, after a certain threshold, could put them more at risk for delirium. In fact, relationship between direct measurement of TNF-alpha level and delirium has been controversial. In patients with delirium, multiple study reported that TNF-alpha remain not significant (de Rooij et al., 2007; Cinar et al., 2014; Brum et al., 2015). Of note, sample sizes of delirium cases in these reports are less than 100 (64 cases, 15 cases, and 17 cases, respectively), thus the result might have been underpowered. Also, controlling for the level of exogenous insults can be difficult confounding factors to control. These conflicting data suggests that cytokine level itself may not work as a reliable biomarker of the risk of delirium, and DNAm may potentially provide a better underline risk factor for delirium.

The TNF-alpha results from the GTP were further interrogated in the NSG cohort, and it was shown that DNAm in almost all CpGs in TNF-alpha decrease with advanced age in blood. Furthermore, DNAm in TNF-alpha CpGs in FACS-sorted neuronal-negative tissue showed a similar decrease with aging. One CpG (cg15989608) was found to be nominally significantly associated with aging even with a limited sample size of 13 cases. This CpG was also significantly associated with aging from blood samples among the GTP cohort with a p-value 5.34×E−5. When the NSG data was compared among six subjects who have samples from whole brain, glia, neuron and blood, a negative correlation was seen for most of TNF-alpha's 24 CpGs in glial, whole brain, and blood tissues, whereas neuronal-positive cells showed a wider range of correlations. This similar trend in glia and blood in TNF-alpha supports our hypothesis that glial cells (including microglia) and blood cells (including monocytes) could have similar DNAm changes in association with aging among pro-inflammatory cytokine genes.

It is possible that the trends we saw in DNAm decreasing with aging were due to the fact that DNAm in general tends to decrease with aging; however, as different tissues varied in their aging trends, this indicates the findings were not generalized. Thus, it is possible that age-associated decreases in DNAm level among pro-inflammatory cytokine genes are more dominant among glia and blood cells, as compared to neuronal cells.

The data presented here provide evidence of DNAm-associated changes in cytokine genes with aging in blood and brain tissues, especially among glial cells. As it is possible that individuals susceptible to delirium have exacerbated or dysregulated changes in DNAm in these genes, this research provides a basis for further testing our hypothesis of epigenetic change in delirium. For this goal, we have initiated a study to collect samples from subjects with and without delirium to test genome-wide DNAm difference.

An identification of epigenetic biomarkers associated with delirium could potentially improve current practice of medicine and surgery. For example, where possible, patients who are identified to be at high risk for delirium may postpone surgery until the risk diminishes, or preventative measure could be employed to have patients closely monitored after surgery to minimize dangerous outcomes. This would allow for limited resources in the hospital to be allocated more efficiently.

In summary, we propose a hypothesis for the role DNAm on cytokine genes in delirium pathophysiology. Specifically, we hypothesize that with aging, there is a decrease in DNAm in pro-inflammatory cytokine genes, which could make them more prone to be expressed, especially in response to exogenous insults, such as infection or surgery. Thus, such inflammatory response with heightened cytokine levels could potentially lead to delirium. This Example showed that DNAm in TNF-alpha and IL-6 CpGs are negatively correlated with aging both in brain and blood tissues. For TNF-alpha, glia and blood showed similar DNAm trends with aging in contrast to neuron tissues. Importantly, this study shows that DNAm levels in cytokine genes are associated with aging, but it is necessary to determine if aged individuals susceptible to delirium have different methylation in contrast to aged individuals who do not develop delirium after exogenous insults.

### Example 2

Given the fact that aging and inflammation are the key risk factors of delirium, we focused on the fact that DNA methylation (DNAm) changes dynamically over the human lifespan and that epigenetic mechanisms control the expression of genes including those of cytokines. Thus, we hypothesized that epigenetic modifications specific to aging and delirium susceptibility occur in microglia; that similar modifications occur in blood; and that these epigenetic changes enhance reactions to exogenous insult, resulting in increased cytokine expression and delirium susceptibility. In fact, no published study has assessed DNAm and its relationship to delirium in humans, especially with genome-wide DNAm investigation.

In this Example we compared DNAm status in blood from hospitalized patients with and without delirium to identify clinically useful epigenetic biomarkers for delirium from blood samples, which are routinely obtained from patients. We used blood for three reasons: 1) the function of monocytes in the blood is similar to that of microglia in that both release cytokines in response to exogenous stimulus, 2) our comparison of DNAm levels in live brain tissue (resected during neurosurgery) to those in blood from same individual collected at the same time point showed a high level of correlation genome-wide, and 3) our previous data showed a similar age-associated decrease in DNAm in the pro-inflammatory cytokine gene TNF-alpha among glia and blood. To accomplish these goals, we conducted the present study investigating DNAm differences in blood between delirium patients and controls without delirium.

To be comprehensive, we employed genome-wide approach using Illumina EPIC array. We first tested the pro-inflammatory cytokine gene, TNF-alpha, and its correlations with age between groups with and without delirium. We also conducted network analysis. Lastly, we compared DNAm age between the two groups.

### Methods

### Study Subjects and Sample Collection

Study participants were co-enrolled when they were enrolled for a separate, ongoing study of delirium. A more detailed overview of study participants' recruitment process has been described previously. Briefly, 92 subjects were recruited for this epigenetics study between November 2017 and October 2018 at the University of Iowa Hospitals and Clinics. This study was approved by the University of Iowa's Human Subjects Research Institutional Review Board.

### Delirium Status Definition

A more detailed overview of study participants' phenotyping has been described previously. Briefly, we screened potential study participants for the presence of delirium by reviewing hospital records and by administering the Confusion Assessment Method for Intensive Care Unit (CAM-ICU), the Delirium Rating Scale-Revised-98 (DRS-R-98), and the Delirium Observation Screening Scale (DOSS). A final decision of delirium category was conducted by detailed chart review by trained psychiatrist (G.S.).

### Sample Processing and Epigenetics Platform

Written informed consent was obtained, and whole blood samples were collected in EDTA tubes. All samples were stored at −80° C. Methylome assays were performed as previously described. Briefly, genomic DNA from whole blood was isolated with the MasterPure™ DNA Purification kit (Epicentre, MCD85201). DNA was bisulfite-converted using the EZ DNA Methylation™ Kit (Zymo Research, D5002). The Infinium HumanMethylationEPIC BeadChip™ Kit (Illumina, WG-317-1002) was used to analyze DNAm of 93 samples from 92 subjects with blood samples (there were two samples from one subject). Raw data was processed using the R packages ChAMP and minfi. Using ChAMP, the samples were filtered out if they had a detection p-value >0.1 (1 sample). The probes were filtered out if they 1) had a detection p-value >0.01 (45,458 probes), 2) had less than 3 beads in at least 5% of samples per probe (8,868 probes), 3) had non-CpG probes (2,761 probes), 4) had SNP-related probes (91,788 probes), 5) had multi-hit probes (11 probes), and 6) located in chromosome X or Y (15,836 probes). After filtering, 92 samples and 701,196 probes remained. Then, quality control and exploratory analysis were conducted. The density plot showed 3 outliers, and multidimensional scaling plot showed 4 outliers. Two of them were in fact from those with two samples from one subject. The other 2 were the same as on the density plot. Total 5 samples were removed, and 87 samples were processed again using ChAMP. The probes were filtered out if they 1) had a detection p-value >0.01 (12,903 probes), 2) had less than 3 beads in at least 5% of samples per probe (8,280 probes), 3) had non-CpG probes (2911 probes), 4) had SNP-related probes (94,425 probes), 5) had multi-hit probes (11 probes), and 6) were located in chromosomes X or Y (16,356 probes). After filtering, 87 samples and 731.032 probes remained. In the quality control and exploratory analysis, the density plot and multidimensional scaling plot showed no outliers. Samples were normalized with beta mixture quantile dilation. Batch effect was corrected using the Combat normalization method as implanted in the package SVA.

### Statistical Analysis

All statistical analyses were performed in R. The correlation of cytokine genes was performed with CpGs from those genes present on the array. The degree of correlation between aging and DNAm was calculated for each CpG in the cytokine genes tested. The correlation coefficient and its significance level were calculated with Pearson's correlation analysis. The categorical data was calculated with chi-square test. DNAm age was calculated using the online DNAm Age Calculator (https://dnamage.genetics.ucla.edu/) after filtering and the quality control process. Cell type proportions of CD8 T cells, CD4 T cells, natural killer cells, B cells, monocytes, and granulocytes were estimated also using the online DNAm Age Calculator (https://dnamage.genetics.ucla.edu/) by using the method described in the previous study. Differential DNA methylation at the level of individual CpGs was analyzed by RnBeads using the limma method. Age, gender, and cell type proportions were included as covariates. Network analysis was conducted using the R package missMethyl by correcting different numbers of probes per gene on the array for Gene Ontology (GO) and Kyoto Encyclopedia of Genes and Genomes (KEGG) enrichment analysis.

### Results

### Comparison of Delirium Cases Vs Non-Delirium Controls in the TNF-Alpha Gene

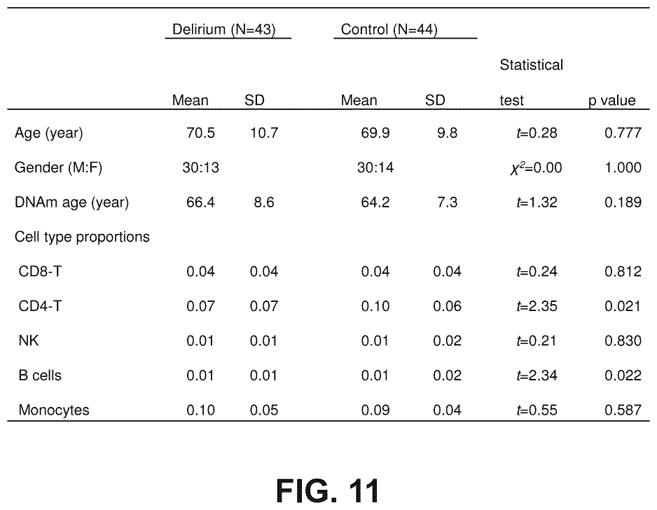

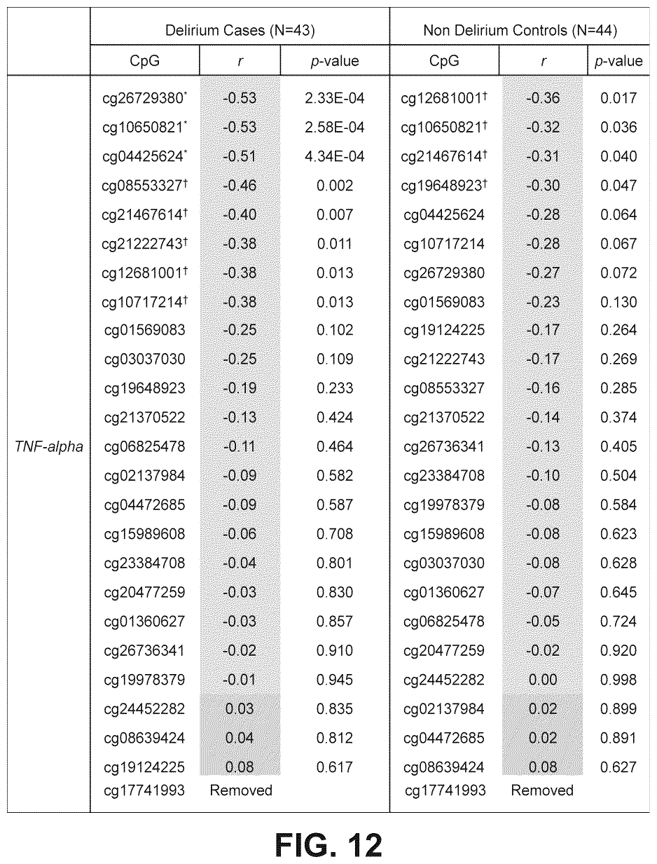

Whole blood samples from 87 samples (43 delirium and 44 non-delirium, age range 42-101 years) after filtering were analyzed using the EPIC array for genome-wide DNAm analysis. There were no significant differences in age, gender, DNAm age, CD8 T cells, natural killer cells, and B cells between delirium cases and non-delirium controls (FIG. 11). Among them, first, we specifically tested correlation between age and DNAm levels at 24 CpGs in the TNF-alpha gene tested on the Illumina EPIC array as shown in FIG. 12. Delirium cases showed 8 nominal significant (p<0.05) CpGs, whereas non-delirium controls showed only 4 nominal significant CpGs. Furthermore, delirium cases showed 3 significant CpGs after correction for multiple testing level (p<0.05/24=0.00208), whereas non-delirium controls showed no significant CpGs after correction for multiple testing level.

### Network Analysis

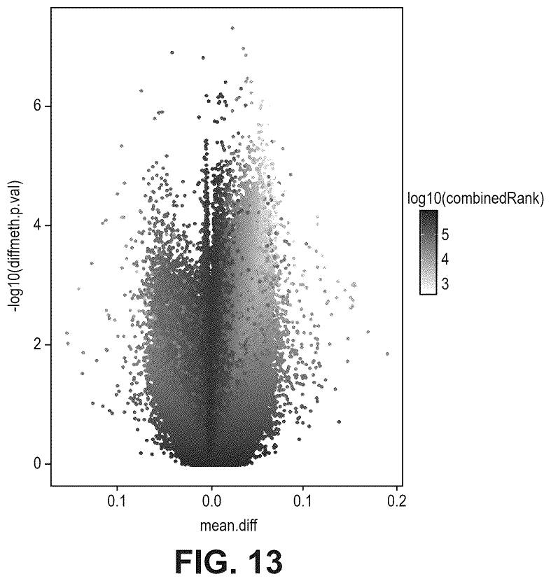

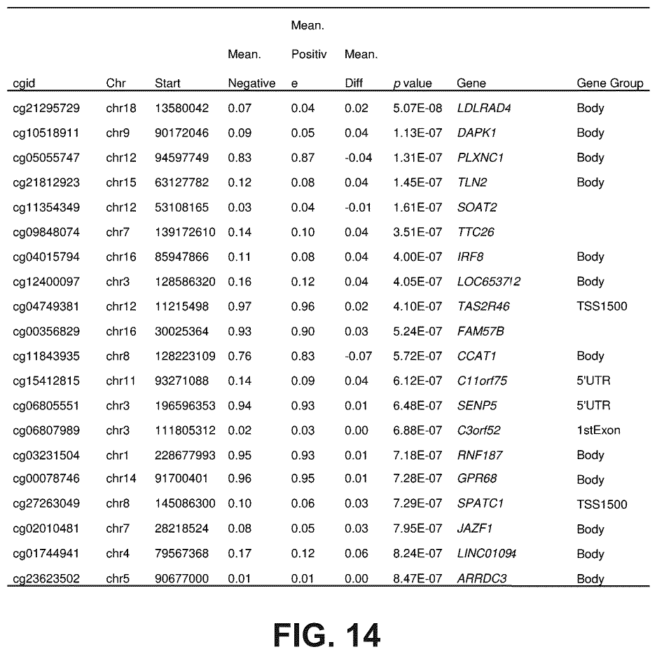

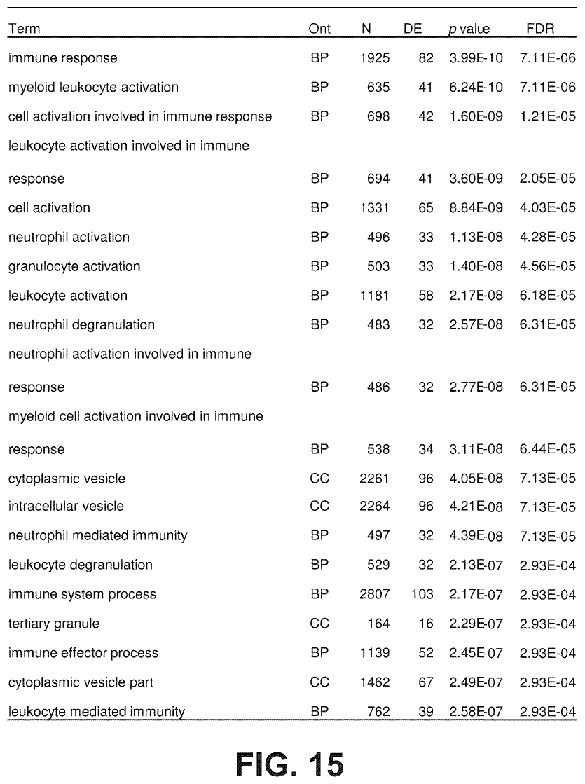

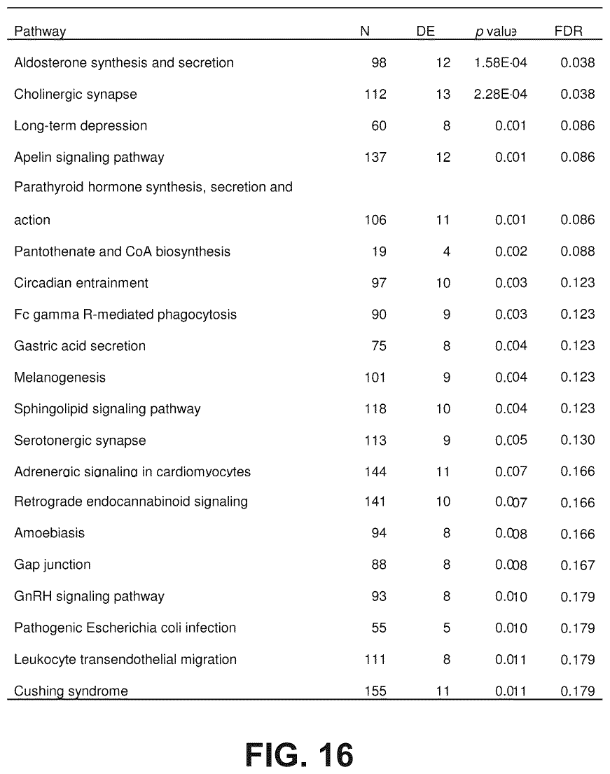

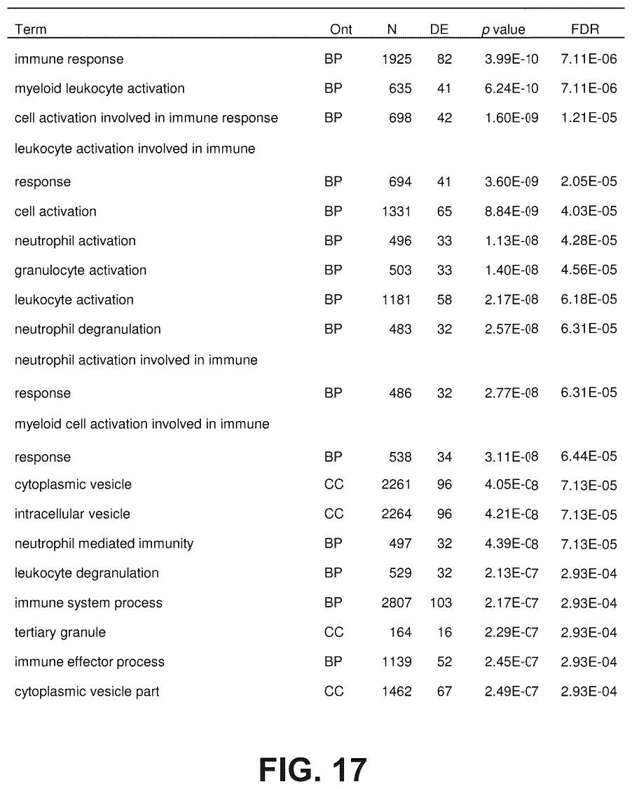

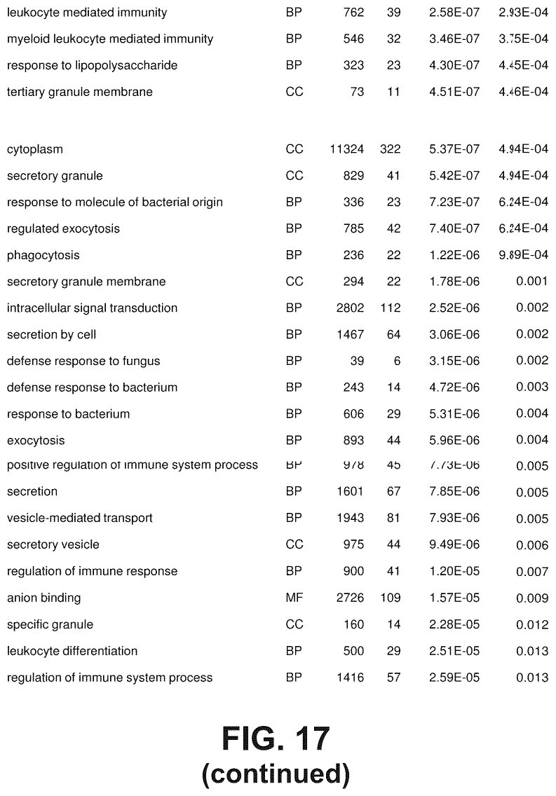

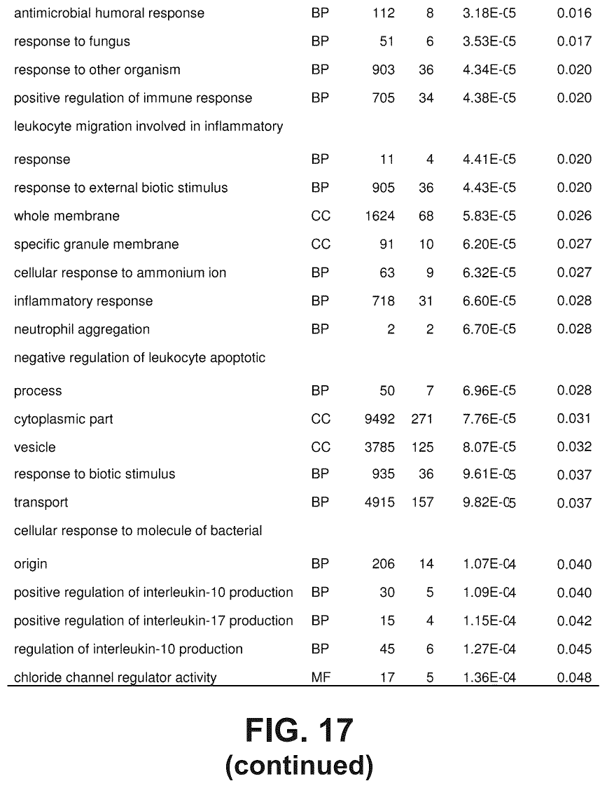

Next, we directly tested genome-wide DNAm differences between delirium cases and non-delirium controls. Volcano plots for the distribution of individual CpG differences and their corresponding logarithmic transformed p-values are shown in FIG. 13. The top 20 differentially methylated CpGs between delirium cases and non-delirium controls are shown in FIG. 14. Genome-wide analysis showed a top hit at cg21295729 with genome-wide significance (p=5.07E-8). This CpG is located near the gene LDLRAD4. Network analysis was conducted by using the CpGs with methylation level differences greater than 5.0% and p-value <0.0005 (n=753). By using those genes, network analysis showed the following results of the top 20 pathways with GO (FIG. 15) and KEGG analysis (FIG. 16). The top pathways with GO were immune response and myeloid leukocyte activation, and with KEGG were aldosterone synthesis/secretion and cholinergic synapse. FIG. 17 also shows the significant pathways of KEGG analysis with false discovery rate (FDR)-adjusted p-value <0.05.

### Comparison of Delirium Cases Vs Non-Delirium Controls: Methylation Age

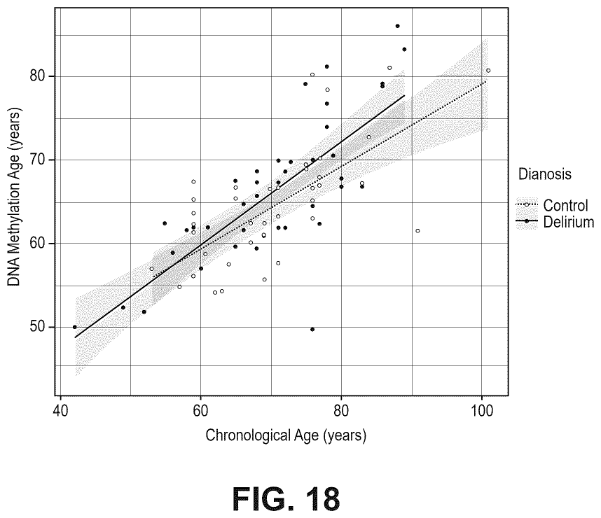

We further tested differences of DNAm age between delirium cases and non-delirium controls. DNAm age showed significant correlation with chronological age among delirium cases (r=0.78, p<0.001) and non-delirium controls (r=0.67, p<0.001). DNAm age among the non-delirium controls showed “slower aging” compared to the delirium cases, although there was no statistically significant difference (FIG. 18).

### DISCUSSION

This is the first study comparing epigenetics status, especially genome-wide DNAm, between patients with and without delirium. The data presented here is consistent with our hypothesis that decreased levels of DNAm on pro-inflammatory cytokines along with aging can lead to heightened inflammation associated with delirium, whereas in patients without delirium DNAm levels remain high, and thus inflammatory reaction could be suppressed and they are protected against delirium.

In the present study, only delirium cases showed significant CpGs with multiple comparison adjusted level between age and decreasing level of TNF-alpha DNAm. This result is consistent with our hypothesis that the DNAm level of pro-inflammatory genes decrease along with aging in delirium patients. We speculate that delirium patients may have persistent decline of DNAm levels in the TNF-alpha gene along with aging, and this may have led to onset of delirium with an additional medical condition, requiring them to be admitted to a hospital. On the other hand, patients without delirium had less decline of DNAm levels in the TNF-alpha gene with aging, thus it might have protected them from developing delirium. To confirm this speculation, we need to conduct a large, prospective study comparing patient population as similar as possible in terms of their medication conditions. Studying surgical patients where you can collect samples before their exposure to surgery would be ideal for such investigation.

From genome-wide DNAm analysis, one genome-wide significant signal in the gene of LDLRAD4 was identified. LDLRAD4—low-density lipoprotein receptor class A domain containing 4—functions as a negative regulator of TGF-beta signaling that regulates the growth, differentiation, apoptosis, motility, and matrix protein production of a lot of cell types. Although it was not genome-wide significant, one of the top hit CpG was near the gene DAPK1. DAPK1—death associated protein kinase 1—functions as regulating cell death, autophagy, and inflammation. The other top hit CpG was near the gene IRF8. IRF8—interferon regulatory factor 8—functions as a modulating of the immune response, cell growth, and oncogenesis.

Although a potential role of these specific genes in pathophysiology of delirium requires further investigation, network analysis identified several top pathways relevant to neurofunction and inflammatory/immune processes, including immune response, leukocyte activation, neutrophil activation, and myeloid cell activation involved in immune response from GO analysis. Many pathways relevant to delirium and neural function were also identified from KEGG analysis, including cholinergic synapse, serotonergic synapse, and leukocyte transendothelial migration. The cholinergic synapse was second from the top KEGG pathways. The cholinergic system is one of the most important neurotransmitter systems in the brain, and deficiency of acetylcholine is well known to be associated with delirium. The results of the present study are further supporting the relevance of cholinergic function in potential pathophysiology of delirium. Furthermore, the pathways of positive regulation and regulation of interleukin-10 production, and positive regulation of interleukin-17 production, were significant (FDR-adjusted p-value <0.05) from GO analysis. This result is also suggesting the role of pro-inflammatory cytokines and neuroinflammation in pathophysiology of delirium, consistent with our hypothesis. These findings may support the validity of this epigenetic investigation of delirium pathophysiology.

We showed that DNAm age was significantly correlated with chronological age in both delirium cases and non-delirium controls. However, non-delirium cases showed relatively slower progression of DNAm aging along with chronological aging than delirium cases. DNAm age measures the accumulative effect of an epigenetic maintenance system and predicts mortality. We speculate that non-delirium controls may have a protective mechanism against DNAm aging, which can also prevent developing delirium. With a larger sample size study we can test if DNAm aging is in fact different between delirium cases versus controls.

In conclusion, to the best of our knowledge, this is the first epigenetics study of delirium. The DNAm was investigated genome-wide. The results were consistent with our previous work and hypothesis. Despite these limitations mentioned above, we showed evidence of epigenetic differences both at gene levels and network levels between delirium cases and non-delirium controls. This finding indicates that DNAm status in blood may become a useful epigenetic biomarker for delirium.

### REFERENCES

1. Inouye S K. Delirium in older persons. N Engl J Med. 2006 Mar. 16; 354(11):1157-65. PubMed PMID: 16540616. Epub 2006 Mar. 17. eng.
2. Spronk P E, Riekerk B, Hofhuis J, Rommes J H. Occurrence of delirium is severely underestimated in the ICU during daily care. Intensive Care Med. 2009 July; 35(7):1276-80. PubMed PMID: 19350214. Pubmed Central PMCID: PMC2698979. Epub 2009 Apr. 8. eng.
3. Pandharipande P P, Girard T D, Jackson J C, Morandi A, Thompson J L, Pun B T, et al. Long-term cognitive impairment after critical illness. N Engl J Med. 2013; 369(14):1306-16.
4. McCusker J. Cole M, Abrahamowicz M, Primeau F, Belzile E. Delirium predicts 12-month mortality. Arch Intern Med. 2002 Feb. 25; 162(4):457-63. PubMed PMID: 11863480. Epub 2002 Feb. 28. eng.
5. Witlox J, Eurelings L S, de Jonghe J F, Kalisvaart K J, Eikelenboom P. Van Gool W A. Delirium in elderly patients and the risk of postdischarge mortality, institutionalization, and dementia: a meta-analysis. JAMA. 2010; 304(4):443-51.
6. Dillon S T, Vasunilashorn S M, Ngo L, Otu H H, Inouye S K, Jones R N, et al. Higher C-Reactive Protein Levels Predict Postoperative Delirium in Older Patients Undergoing Major Elective Surgery: A Longitudinal Nested Case-Control Study. Biol Psychiatry. 2017 Jan. 15; 81(2):145-53. PubMed PMID: 27160518. Pubmed Central PMCID: PMC5035711. Epub 2016 May 11. eng.
7. Vasunilashorn S M, Dillon S T, Inouye S K, Ngo L H, Fong T G, Jones R N, et al. High C-Reactive Protein Predicts Delirium Incidence, Duration, and Feature Severity After Major Noncardiac Surgery. J Am Geriatr Soc. 2017 August; 65(8):e109-e16. PubMed PMID: 28555781. Pubmed Central PMCID: PMC5700456. Epub 2017 May 31. eng.
8. Zhu Y, Hu W. Zhu M L, Yin T, Su J, Wang J R. Serum galectin-3 levels and delirium among postpartum intensive care unit women. Brain Behav. 2017 August; 7(8):e00773. PubMed PMID: 28828226. Pubmed Central PMCID: PMC5561326. Epub 2017 Aug. 23. eng.
9. Vasunilashorn S M, Ngo L, Inouye S K, Libermann T A, Jones R N, Alsop D C, et al. Cytokines and Postoperative Delirium in Older Patients Undergoing Major Elective Surgery. J Gerontol A Biol Sci Med Sci. 2015 October; 70(10):1289-95. PubMed PMID: 26215633. Pubmed Central PMCID: PMC4817082. Epub 2015 Jul. 29. eng.
10. de Rooij S E, van Munster B C, Korevaar J C, Levi M. Cytokines and acute phase response in delirium. J Psychosom Res. 2007: 62 (5): 521-5.
11. Khan B A, Zawahiri M, Campbell N L, Boustani M A. Biomarkers for delirium—a review. J Am Geriatr Soc. 2011:59:S256-S61.
12. Hovens I B, van Leeuwen B L, Nyakas C, Heineman E, van der Zee E A, Schoemaker R G. Postoperative cognitive dysfunction and microglial activation in associated brain regions in old rats. Neurobiol Learn Mem. 2015 February; 118:74-9. PubMed PMID: 25460037. Epub 2014 Dec. 3. eng.
13. Chen J, Buchanan J B, Sparkman N L, Godbout J P, Freund G G, Johnson R W.

Neuroinflammation and disruption in working memory in aged mice after acute stimulation of the peripheral innate immune system. Brain Behav Immun. 2008 March; 22(3):301-11. PubMed PMID: 17951027. Pubmed Central PMCID: PMC2374919. Epub 2007 Oct. 24. eng.
14. Xu Z, Dong Y, Wang H, Culley D J, Marcantonio E R, Crosby G, et al. Peripheral surgical wounding and age-dependent neuroinflammation in mice. PLOS One. 2014; 9(5):e96752. PubMed PMID: 24796537. Pubmed Central PMCID: PMC4010504. Epub 2014 May 7. eng.
15. Wang H L, Ma R H, Fang H, Xue Z G, Liao Q W. Impaired Spatial Learning Memory after Isoflurane Anesthesia or Appendectomy in Aged Mice is Associated with Microglia Activation. J Cell Death. 2015; 8:9-19. PubMed PMID: 26380557. Pubmed Central PMCID: PMC4560456. Epub 2015 Sep. 19. eng.
16. Shinozaki G, Braun P R, Hing B W Q. Ratanatharathorn A, Klisares M J, Duncan G N, et al. Epigenetics of Delirium and Aging: Potential Role of DNA Methylation Change on Cytokine Genes in Glia and Blood Along With Aging. Front Aging Neurosci. 2018:10:311. PubMed PMID: 30405391. Pubmed Central PMCID: PMC6206747. Epub 2018 Nov. 9. eng.
17. Braun P R, Han S, Hing B, Nagahama Y, Gaul L N, Heinzman J T, et al. Genome-wide DNA methylation comparison between live human brain and peripheral tissues within individuals. Translational psychiatry. 2019; 9(1):47.
18. Shinozaki G, Chan A C, Sparr N A, Zarei K, Gaul L N, Heinzman J T, et al. Delirium detection by a novel bispectral electroencephalography device in general hospital. Psychiatry Clin Neurosci. 2018 December; 72(12):856-63. PubMed PMID: 30246448. Epub 2018 Sep. 25. eng.
19. Ely E W, Inouye S K, Bernard G R, Gordon S, Francis J, May L, et al. Delirium in mechanically ventilated patients: validity and reliability of the confusion assessment method for the intensive care unit (CAM-ICU). JAMA. 2001 Dec. 5; 286(21):2703-10. PubMed PMID: 11730446. Epub 2001 Dec. 26. eng.
20. Trzepacz P T, Mittal D, Torres R, Kanary K. Norton J, Jimerson N. Validation of the Delirium Rating Scale-revised-98: comparison with the delirium rating scale and the cognitive test for delirium. J Neuropsychiatry Clin Neurosci. 2001 Spring: 13(2):229-42. PubMed PMID: 11449030. Epub 2001 Jul. 13. eng.
21. Schuurmans M J, Shortridge-Baggett L M, Duursma S A. The Delirium Observation Screening Scale: a screening instrument for delirium. Res Theory Nurs Pract. 2003 Spring: 17(1):31-50. PubMed PMID: 12751884. Epub 2003 May 20. eng.
22. Morris T J, Butcher L M, Feber A, Teschendorff A E, Chakravarthy A R, Wojdacz T K, et al. ChAMP: 450k Chip Analysis Methylation Pipeline. Bioinformatics. 2014 Feb. 1; 30(3):428-30. PubMed PMID: 24336642. Pubmed Central PMCID: PMC3904520. Epub 2013 Dec. 18. eng.
23. Aryee M J, Jaffe A E, Corrada-Bravo H, Ladd-Acosta C, Feinberg A P, Hansen K D, et al. Minfi: a flexible and comprehensive Bioconductor package for the analysis of Infinium DNA methylation microarrays. Bioinformatics. 2014; 30(10):1363-9.
24. Fortin J P. Triche T, Hansen K. Preprocessing, normalization and integration of the Illumina HumanMethylationEPIC array. BioRxiv. 2016:065490.
25. Zhou W. Laird P W, Shen H. Comprehensive characterization, annotation and innovative use of Infinium DNA methylation BeadChip probes. Nucleic Acids Res. 2017; 45(4):e22-e.
26. Nordlund J, Bäcklin C L, Wahlberg P, Busche S, Berglund E C, Eloranta M L, et al. Genome-wide signatures of differential DNA methylation in pediatric acute lymphoblastic leukemia. Genome Biol. 2013; 14(9):r105.
27. Teschendorff A E, Marabita F, Lechner M, Bartlett T, Tegner J, Gomez-Cabrero D, et al. A beta-mixture quantile normalization method for correcting probe design bias in Illumina Infinium 450 k DNA methylation data. Bioinformatics. 2013 Jan. 15; 29(2):189-96. PubMed PMID: 23175756. Pubmed Central PMCID: PMC3546795. Epub 2012 Nov. 24. eng.
28. Johnson W E, Li C, Rabinovic A. Adjusting batch effects in microarray expression data using empirical Bayes methods. Biostatistics. 2007 January; 8(1):118-27. PubMed PMID: 16632515. Epub 2006 Apr. 25. eng.
29. Leek J T, Johnson W E, Parker H S, Fertig E J, Jaffe A E, Storey J D, et al. sva: Surrogate variable analysis. R package version. 2019.
30. R: A language and environment for statistical computing [Internet]. R Foundation for Statistical Computing. 2019 [cited accessed 23 Apr. 2019]. Available from: https://www.R-project.org/.
31. Horvath S. DNA methylation age of human tissues and cell types. Genome Biol. 2013: 14(10):3156.
32. Houseman E A, Accomando W P, Koestler D C, Christensen B C, Marsit C J, Nelson H H, et al. DNA methylation arrays as surrogate measures of cell mixture distribution. BMC Bioinformatics. 2012; 13(1):86.
33. Assenov Y, Müller F, Lutsik P, Walter J. Lengauer T. Bock C. Comprehensive analysis of DNA methylation data with RnBeads. Nature methods. 2014; 11(11):1138.
34. Ritchie M E, Phipson B, Wu D, Hu Y, Law C W, Shi W, et al. limma powers differential expression analyses for RNA-sequencing and microarray studies. Nucleic Acids Res. 2015: 43(7):e47-e.
35. Phipson B, Maksimovic J, Oshlack A. missMethyl: an R package for analyzing data from Illumina's HumanMethylation450 platform. Bioinformatics. 2016 Jan. 15; 32(2):286-8. PubMed PMID: 26424855. Epub 2015 Oct. 2. eng.
36. Nakano N, Maeyama K, Sakata N, Itoh F. Akatsu R, Nakata M, et al. C18 ORF1, a novel negative regulator of transforming growth factor-β signaling. J Biol Chem. 2014; 289(18):12680-92.
37. Yuan W, Chen J, Shu Y, Liu S, Wu L, Ji J, et al. Correlation of DAPK1 methylation and the risk of gastrointestinal cancer: A systematic review and meta-analysis. PLOS One. 2017; 12(9):e0184959. PubMed PMID: 28934284. Pubmed Central PMCID: PMC5608298. Epub 2017 Sep. 22. eng.
38. Li X. Pu J, Liu J, Chen Y, Li Y, Hou P, et al. The prognostic value of DAPK1 hypermethylation in gliomas: A site-specific analysis. Pathol Res Pract. 2018 July; 214(7):940-8. PubMed PMID: 29807777. Epub 2018 May 29. eng.
39. Qiu Y, Yu H, Zhu Y. Ye Z. Deng J, Su W, et al. Hypermethylation of Interferon Regulatory Factor 8 (IRF8) Confers Risk to Vogt-Koyanagi-Harada Disease. Sci Rep. 2017 Apr. 21; 7(1):1007. PubMed PMID: 28432342. Pubmed Central PMCID: PMC5430771. Epub 2017 Apr. 23. eng.
40. Hshieh T T, Fong T G, Marcantonio E R, Inouye S K. Cholinergic deficiency hypothesis in delirium: a synthesis of current evidence. The Journals of Gerontology Series A: Biological Sciences and Medical Sciences. 2008; 63(7):764-72.
41. Maldonado J R. Neuropathogenesis of delirium: review of current etiologic theories and common pathways. The American Journal of Geriatric Psychiatry. 2013; 21(12):1190-222.
42. Christiansen L, Lenart A, Tan Q, Vaupel J W. Aviv A, McGue M, et al. DNA methylation age is associated with mortality in a longitudinal Danish twin study. Aging Cell. 2016; 15(1):149-54.
43. Marioni R E, Shah S, McRae A F, Chen B H, Colicino E, Harris S E, et al. DNA methylation age of blood predicts all-cause mortality in later life. Genome Biol. 2015; 16(1):25.

### FIGURE Captions

Supplementary Table 1 (FIG. 11) Demographic data of delirium cases and non-delirium controls. Abbreviation: DNAm; DNA methylation, CD8-T; CD8 T cells, CD4-T; CD4 T cells, NK; natural killer cells.

Table 1 (FIG. 12) Correlation of age and blood DNAm at 24 CpGs in the TNF-alpha gene compared between delirium cases vs non-delirium controls. Notes: Pink highlights a negative correlation, and blue highlights a positive correlation. *Significant after correction for multiple testing level (p<0.05/24), ^{†}nominally significant (p<0.05).

FIG. 1 (FIG. 13) Volcano plots for the distribution of individual CpG differences and their corresponding logarithmic transformed p-values (n=87)

Supplementary Table 2 (FIG. 14) Top 20 differentially methylated CpGs between delirium cases and non-delirium controls

Table 2 (FIG. 15) Result of the top 20 pathways of GO analysis with CpGs differentially methylated between delirium cases and non-delirium controls. Abbreviations: GO; Gene Ontology, FDR: false discovery rate.

Table 3 (FIG. 16) Result of the top 20 pathways of KEGG analysis with CpGs differentially methylated between delirium cases and non-delirium controls. Abbreviations: KEGG; Kyoto Encyclopedia of Genes and Genomes, FDR; false discovery rate.

Supplementary Table 3 (FIG. 17) Result of the significant pathways of GO analysis with CpGs differentially methylated between delirium cases and non-delirium controls. Abbreviation: KEGG: Kyoto Encyclopedia of Genes and Genomes, FDR; false discovery rate.

FIG. 2 (FIG. 18) Correlation between chronological age and DNAm age in delirium (n=43) vs controls (n=44). Abbreviation: DNAm; DNA methylation

### Example 3

Although delirium is common among elderly patients, it is underdiagnosed and undertreated. Various screening methods have been developed to characterize the epidemiology and risk factors of delirium (e.g., the Confusion Assessment Method (CAM) and Confusion Assessment Method for Intensive Care Unit (CAM-ICU)). Although they have excellent sensitivity and specificity in research settings, they are found to have suboptimal sensitivity (38-47%) in the context of “real world” intensive care units. Thus, among elderly patients at high risk for delirium, identification of biomarkers of delirium would aid in diagnosis and following intervention. Above, we reported the potential role of epigenetics, especially DNA methylation (DNAm) in pro-inflammatory cytokine genes, in pathophysiology of delirium, highlighting the role of neuroinflammation. However, given the complexity of pathophysiological mechanism of delirium as well as known risk factor of delirium among dementia patients, it is important to examine other factors such as neurotrophic factors which are strongly associated with age-related cognitive function, as well.

Among various genes associated with cognitive function, brain-derived neurotrophic factor (BDNF) and glial cell-derived neurotrophic factor (GDNF) are signaling molecules contributing to neural plasticity, learning, and memory. Decreased serum level of BDNF is associated with cognitive impairment. The knockdown model of Neuronal Per-Arnt-Sim domain protein 4 (NPAS4) causes increasing cell death. In-vivo investigation in knockdown model shows increased lesion size and neurodegeneration after photochemically-induced stroke. The nuclear receptor subfamily 4A2 (NR4A2) critically regulates Alzheimer's disease (AD)-related pathophysiology. NR4A2 knockdown mouse model exacerbates AD symptoms/pathology (neuroinflammation/degeneration, plaque accumulation). Administration of FDA-approved NR4A2 modulating drug significantly reduces cognitive impairments and reduces plaques.

Aging alters gene expression in the brain drastically and such changes are precisely regulated by a variety of epigenetic modifications including: DNA methylation (DNAm), histone modifications, and micro RNA-mediated transcriptional control. Indeed, DNAm patterns have been shown to change dynamically throughout the human lifespan, and epigenetic processes involved in aging have been studied in detail. In regards to neurotrophic genes, DNAm of BDNF in blood is significantly correlated with aging in previous study. However, no studies have investigated the role of DNAm of neurotrophic genes in delirium. Our aim was to examine DNAm in neurotrophic factors and investigate whether DNAm of neurotrophic factors correlates with aging, and determine whether such trends are consistent between brain and blood samples and whether there are any difference between delirium patients and those without delirium.

In this study, we analyzed samples from 3 different cohorts to examine correlation between DNAm and age. Specifically, we investigated association between aging and DNAm levels at CpGs in neurotrophic genes, and tested if results could be replicated in these cohorts. We hypothesized that DNAm in neurotrophic genes in both brain and blood increases with aging suggesting less expression of neurotrophic factors, and such changes are more significant among delirium patients.

### Methods

Samples from Grady Trauma Project (GTP) Cohort

Three hundred and eighty-three subjects were analyzed from the Grady Trauma Project (GTP) cohort. The detailed information can be found in the previous study (Shinozaki, Braun, et al., 2018). DNAm data was processed by using Illumina HumanMethylation450. The pipeline employed was described in detail elsewhere.

Samples from Neurosurgery (NSG) Cohort

Twenty-one subjects were analyzed from the Neurosurgery (NSG) cohort. The detailed information was described in the previous study (Braun et al., 2019). Briefly, the subjects with intractable epilepsy undergoing neurosurgery were participated between March 2014 and April 2017 at the University of Iowa Hospitals and Clinics. Written informed consent was obtained from all of the participants. This study was approved by the University of Iowa's Human Subjects Research Institutional Review Board. Whole blood were collected with EDTA tubes, saliva with the Oragene DISCOVER™ kit (DNA Genotek Inc., OGR-500), and buccal tissue with swabs (Puritan, 25-1506 1 PF TT MC). Genomic DNA was extracted with the MasterPure™ DNA Purification kit (Epicentre, MCD85201) from saliva, buccal, and whole blood. Then, DNA was bisulfite-converted with the EZ DNA Methylation™ Kit (Zymo Research, D5002). DNAm was analyzed with the Infinium HumanMethylationEPIC BeadChip™ Kit (Illumina, WG-317-1002). The R packages RnBeads and Minfi were used to process the raw data, quality control checks, data filtering, normalization of the data, and differential methylation analyses.

Subjects and Samples from Epigenetics of Delirium (EOD) Cohort

Ninety-two subjects were participated when they were enrolled for a separate ongoing study of delirium. The detailed information can be found in the previous study (Shinozaki et al., 2019; Shinozaki, Braun, et al., 2018; Shinozaki, Chan, et al., 2018). Briefly, the subjects were participated between November 2017 and October 2018 at the University of Iowa Hospitals and Clinics. Written informed consent was obtained from all of the participants. This study was approved by the University of Iowa's Human Subjects Research Institutional Review Board.

### Definition of Delirium Status

The detailed information can be found in the previous study (Shinozaki, Chan, et al., 2018). Briefly, participants were screened for delirium by hospital records, the Confusion Assessment Method for Intensive Care Unit (CAM-ICU, the Delirium Rating Scale-Revised-98 (DRS-R-98), and the Delirium Observation Screening Scale (DOSS). A final decision of delirium phenotyping was conducted by trained psychiatrist.

### Sample Processing

Whole blood samples were collected with EDTA tubes and all samples were stored at −80° C. Methylome assays were processed as previously described. Briefly, genomic DNA was extracted with the MasterPure™ DNA Purification kit (Epicentre, MCD85201) from whole blood. Then, DNA was bisulfite-converted with the EZ DNA Methylation™ Kit (Zymo Research, D5002).

### Epigenetics Platform

The Infinium HumanMethylationEPIC BeadChip™ Kit (Illumina, WG-317-1002) was used to analyze DNAm of 93 samples from 92 subjects with blood samples (there were 2 samples from 1 subject). The R packages ChAMP and minfi were used to process the raw data. The samples were filtered out if they had a detection p-value more than 0.1 (there was 1 sample), and the probes were filtered out if they 1) had a detection p-value more than 0.01 (there were 45,458 probes), 2) had less than 3 beads in at least 5% of samples per probe (there were 8,868 probes), 3) had non-CpG probes (there were 2,761 probes), 4) had SNP-related probes (there were 91,788 probes), 5) had multi-hit probes (there were 11 probes), and 6) located in chromosome X or Y (there were 15,836 probes) with the ChAMP package. Ninety-two samples and 701,196 probes remained after above filtering. In quality control and exploratory analysis, the density plot showed 3 outliers, and multidimensional scaling plot showed 4 outliers (two of them were from 1 subject, and the other 2 were the same as on the density plot and multidimensional scaling plot). Totally, 5 samples were removed and 87 samples were processed again with the ChAMP package. The probes were filtered out if they 1) had a detection p-value more than 0.01 (there were 12,903 probes), 2) had less than 3 beads in at least 5% of samples per probe (there were 8,280 probes), 3) had non-CpG probes (there were 2911 probes), 4) had SNP-related probes (there were 94,425 probes), 5) had multi-hit probes (there were 11 probes), and 6) were located in chromosomes X or Y (there were 16,356 probes). After filtering, 87 samples and 731,032 probes remained. In quality control and exploratory analysis, the density plot and multidimensional scaling plot showed no outliers. Samples were normalized with beta mixture quantile dilation. Batch effect was corrected with the Combat normalization method as implanted in the package SVA.

### Statistical Analysis

All statistical analyses were performed with R (R Core Team, 2019). The correlation between age and DNAm levels of neurotrophic genes in each CpGs were tested by Pearson's correlation analysis. Genome-wide significant level was determined as p<5×E−8, and nominally significant level as p<0.05.

### Results

DNA Methylation and Expression from GTP Cohort Blood Samples

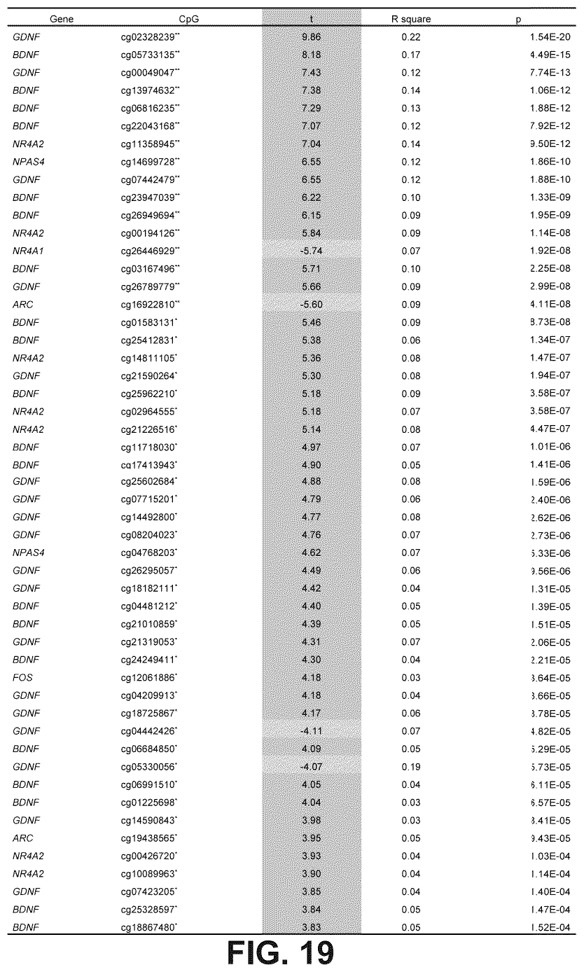

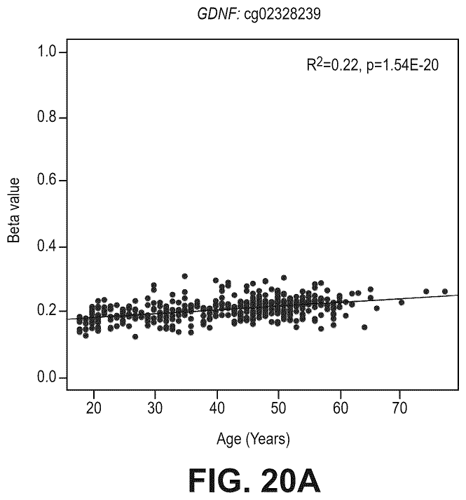

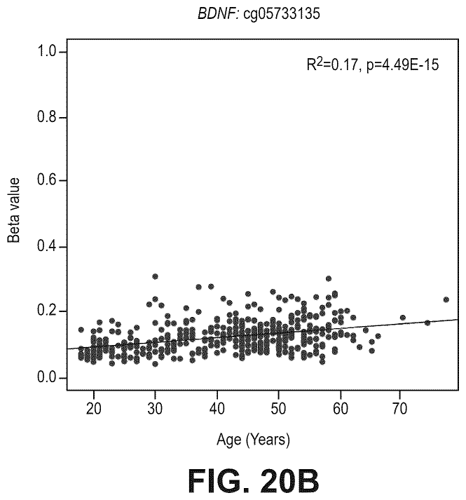

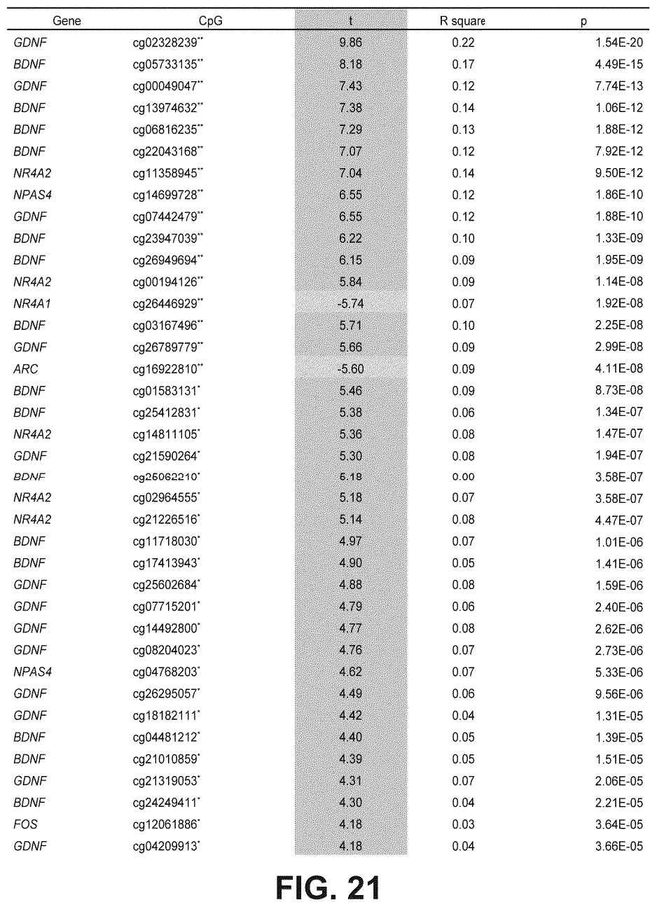

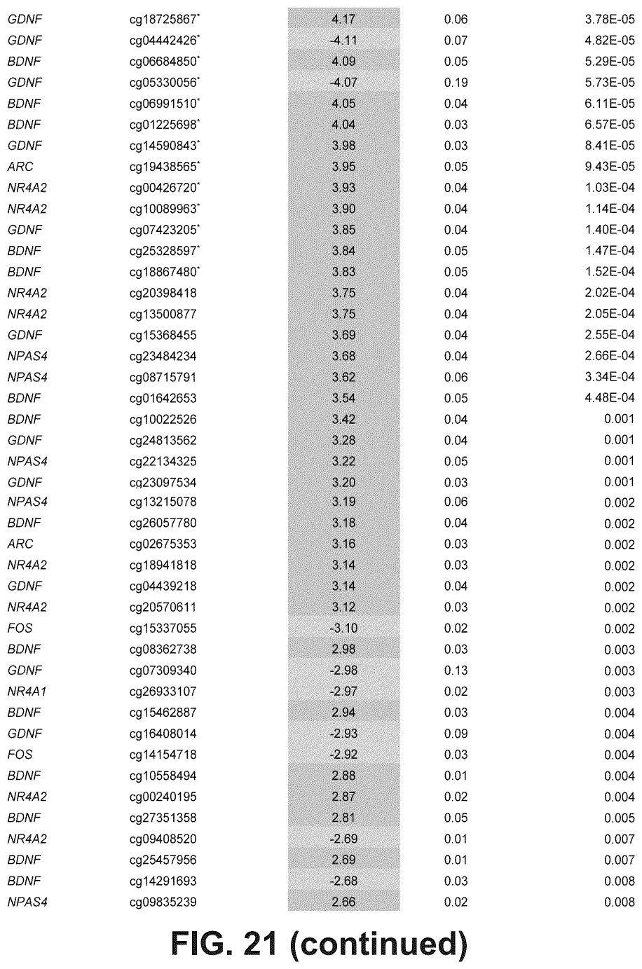

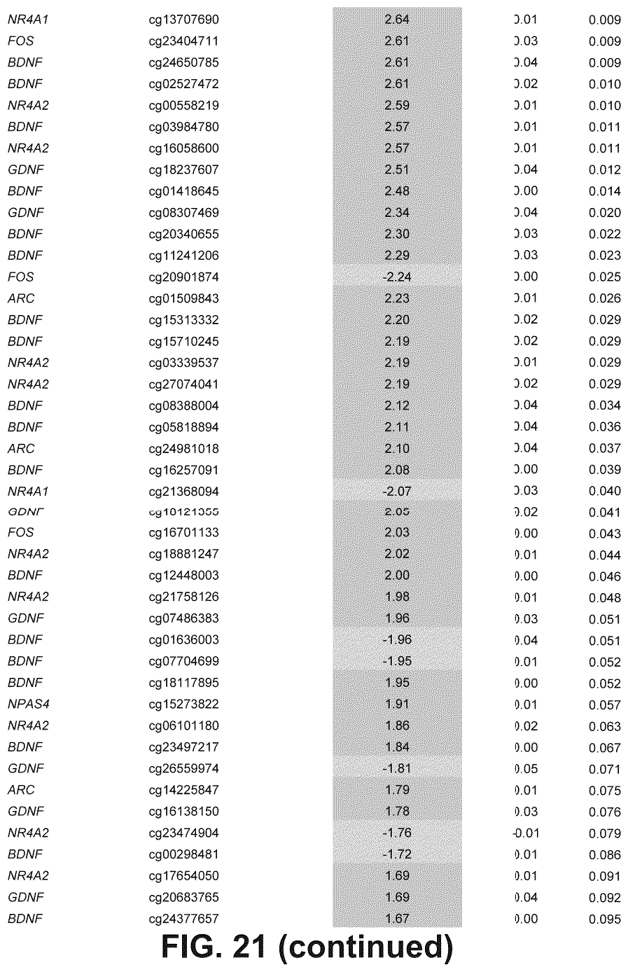

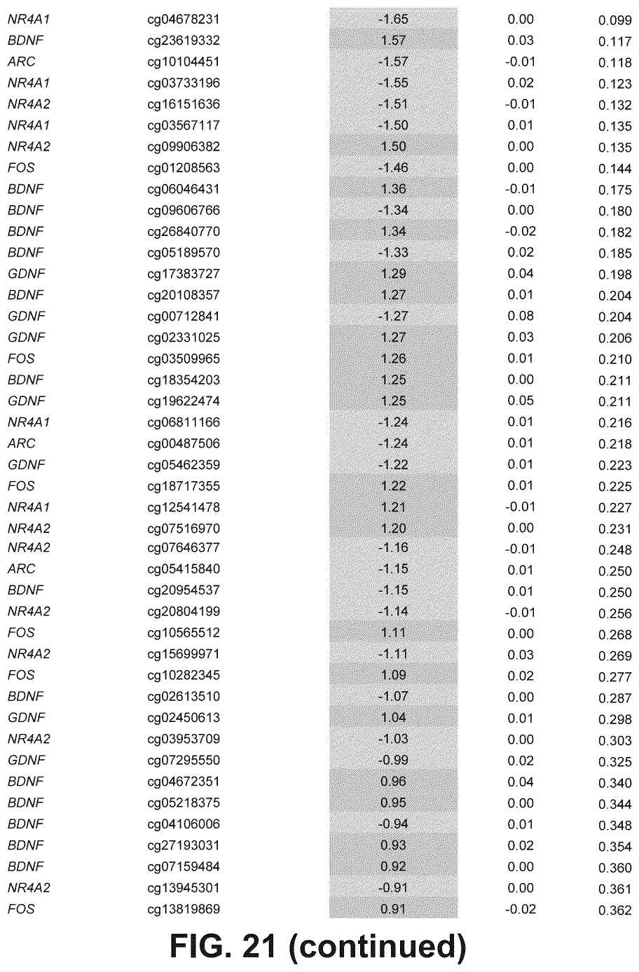

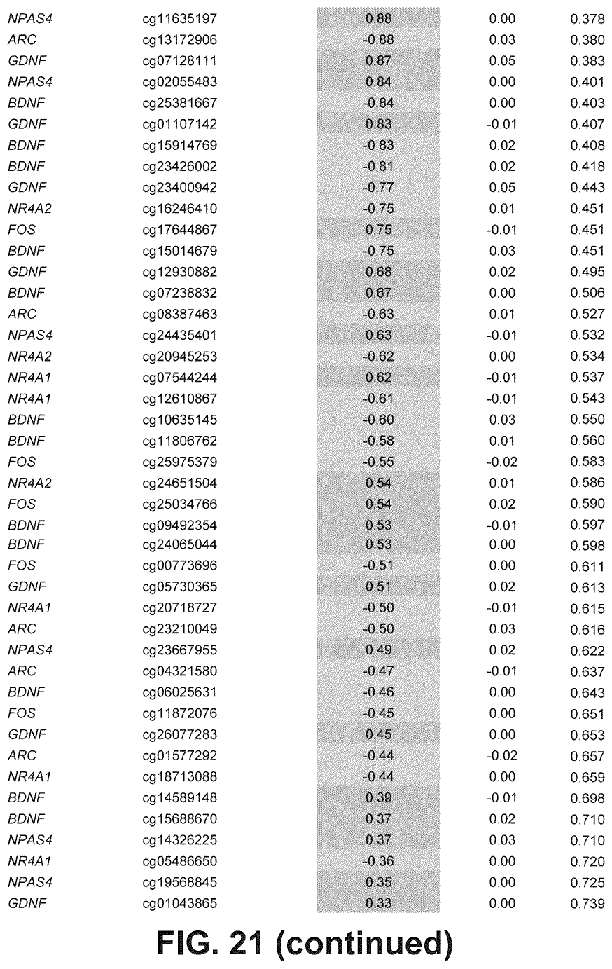

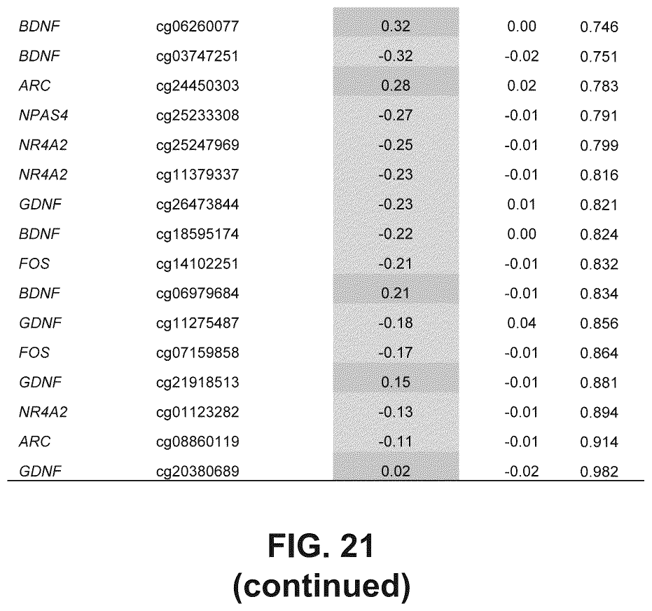

To test the concept underlying this study that DNAm patterns among neurotrophic genes correlate with aging, we first used a dataset based on blood samples from 383 subjects (age average=41.50, age SD=12.93, age range=18-77, female N=273, Race=380 African American, 2 Native American and 1 other). We investigated the association between aging and DNAm levels at CpGs in 7 neurotrophic genes; BDNF, GDNF, activity regulated cytoskeleton associated protein (ARC), Fos Proto-Oncogene, AP-1 Transcription Factor Subunit (FOS), NPAS4, nuclear receptor subfamily 4A1 (NR4A1) and NR4A2. This dataset included total 226 CpGs across those genes, among which DNAm levels at 109 CpGs were associated with aging at nominal significance (p<0.05). In fact, 16 CpGs were significant at Bonferroni corrected genome-wide significance levels (p<5×E−8), mainly from BDNF (7 CpGs) and GDNF (4 CpGs). The top hit was at cg02328239 in GDNF (p=1.54×E−20), and second top was at cg05733135 in BDNF (p=4.49×E−15) (FIGS. 19, 20A, 20B). Among top 53 CpGs with p<2×E−4 (Bonferroni corrected for 226 CpGs, significant level p<0.05/226), 49 CpGs (92.5%) were positively correlated with aging (FIG. 20). Correlations between age and DNAm levels at 226 CpGs were shown in FIG. 21. However, testing of the expression levels of these genes in blood samples showed that expression did not have a significant association with aging (Data not shown).

### NSG Study Samples, DNAm in Brain

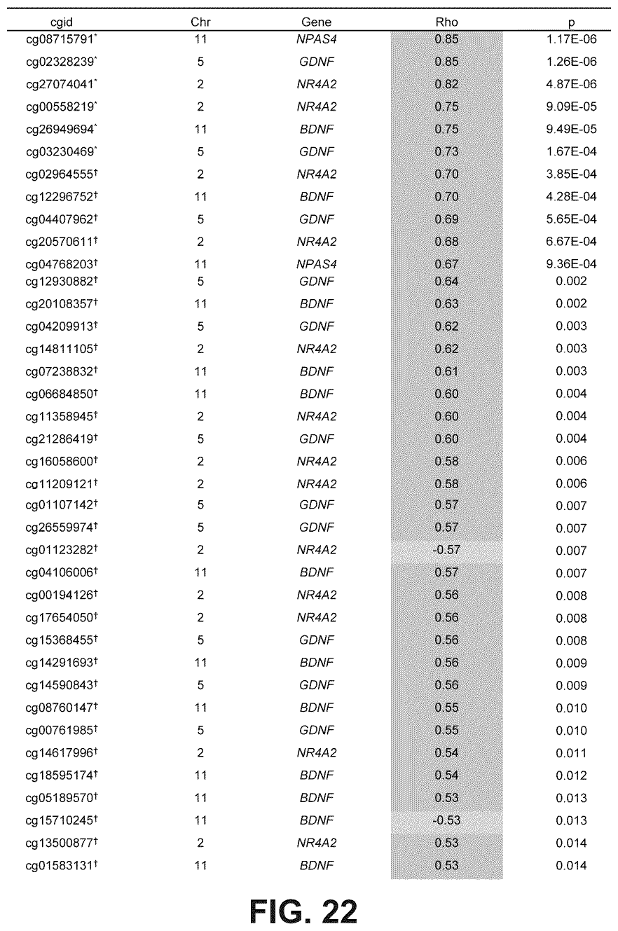

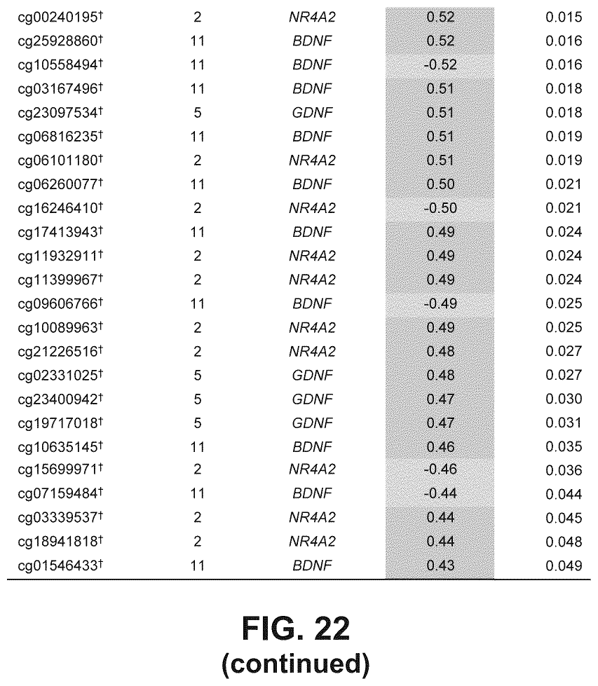

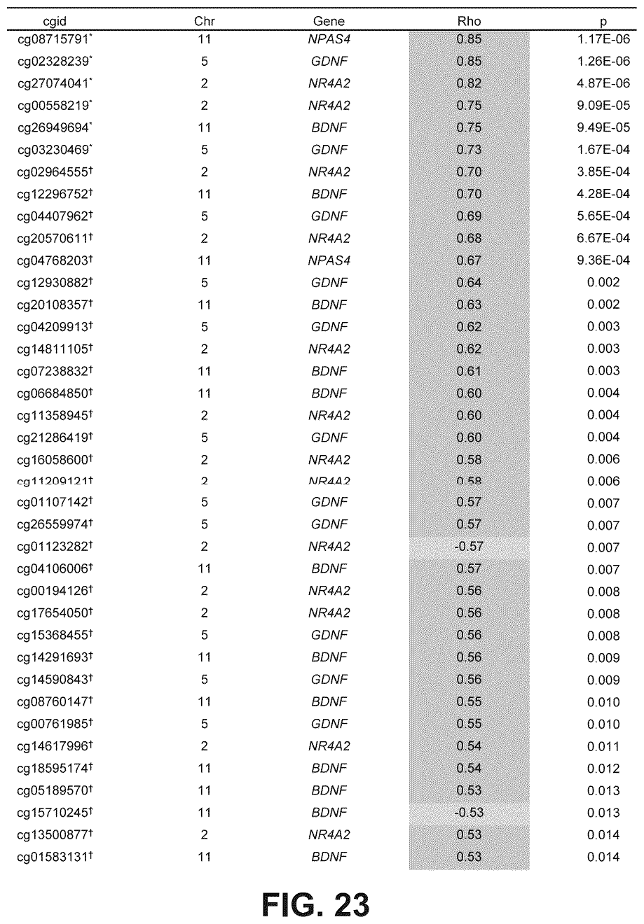

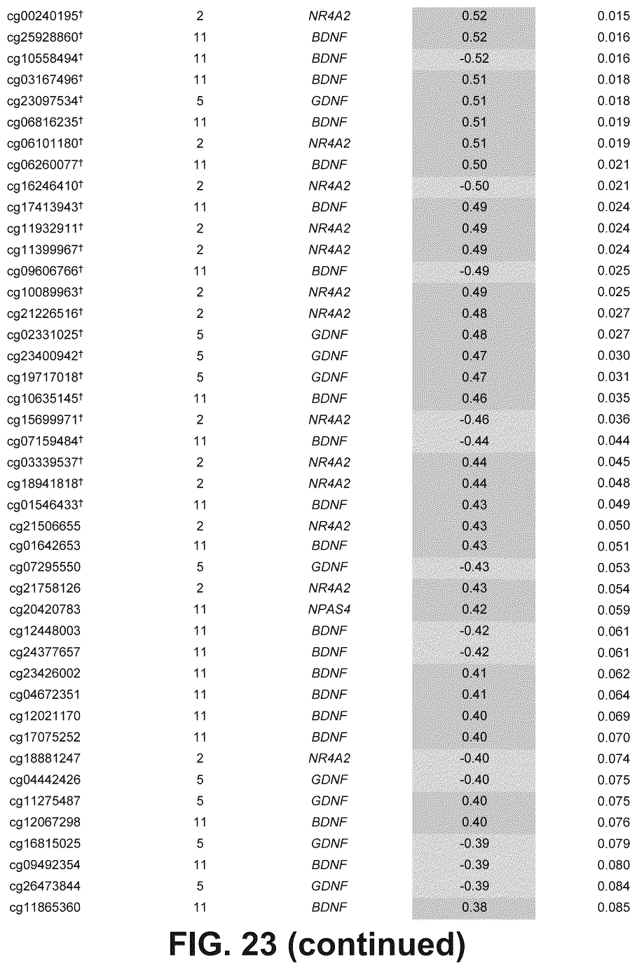

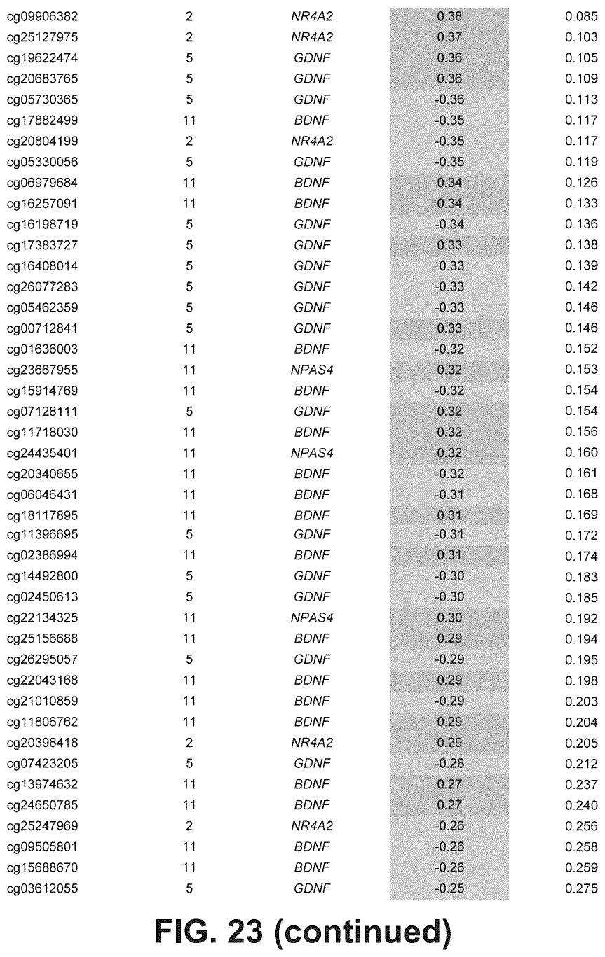

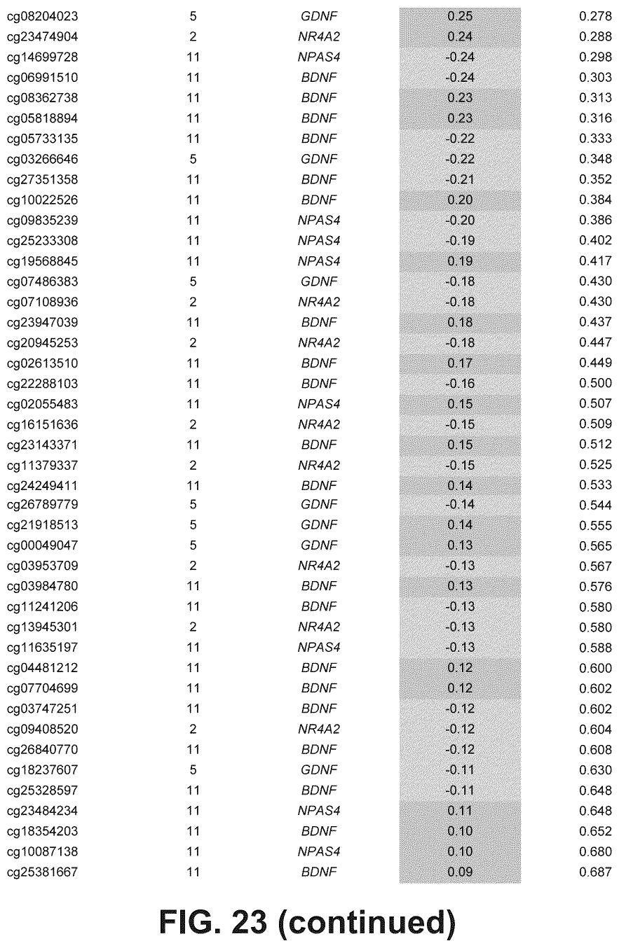

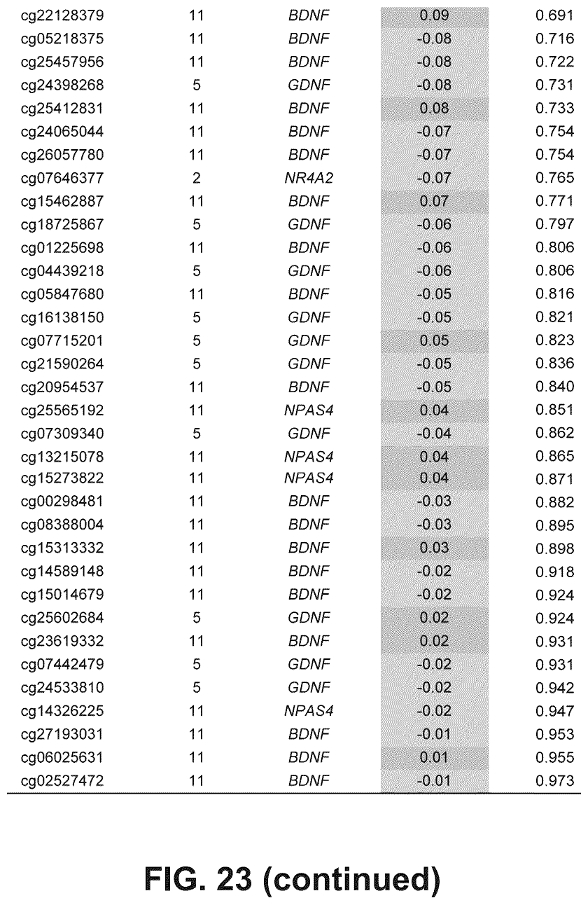

We next tested whether these associations of DNAm in the GTP cohort were also present in brain tissue in our NSG cohort (N=21). We focused on top 4 genes in the GTP cohort (BDNF, GDNF, NR4A2, and NPAS4). In BDNF. 18 CpGs were positively correlated with aging at nominal significance (p<0.05), whereas 4 CpGs were negatively correlated with aging at nominal significance (Table 2). Similarly, 15 CpGs were positively correlated with aging at nominal significance, whereas no CpGs were negatively correlated with aging at nominal significance in GDNF. 20 CpGs were positively correlated with aging at nominal significance, whereas 3 CpGs were negatively correlated with aging at nominal significance in NR4A2, and 2 CpGs were positively correlated with aging at nominal significance, whereas no CpGs were negatively correlated with aging at nominal significance in NPAS4. Furthermore, the top hit CpG at cg02328239 in GDNF in the GTP study (p=1.54×E−20) was also positively correlated with aging with p=1.26×E−6 at multiple testing significant level (Bonferroni corrected for 201 CpGs, significant level p<0.05/201=2.49×E−4) in brain tissue in the NSG study (FIG. 22). In fact, among the nominal significant (p<0.05) CpGs in brain tissue in the NSG study, 3 CpGs in BDNF, 3 CpGs in GDNF. 3 CpGs in NR4A2, and 2 CpGs in NPAS4 were also nominally significant in the GTP study (FIG. 19, 22). Correlations between age and DNAm levels at 201 CpGs were shown in FIG. 23.

### Comparison of Delirium Cases Vs Non-Delirium Controls in EOD Study Samples

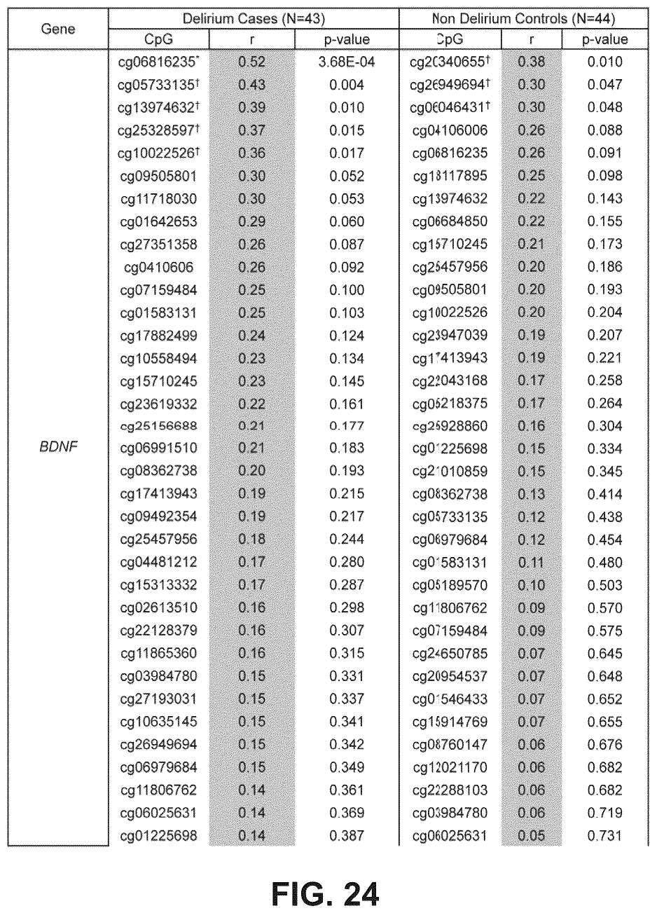

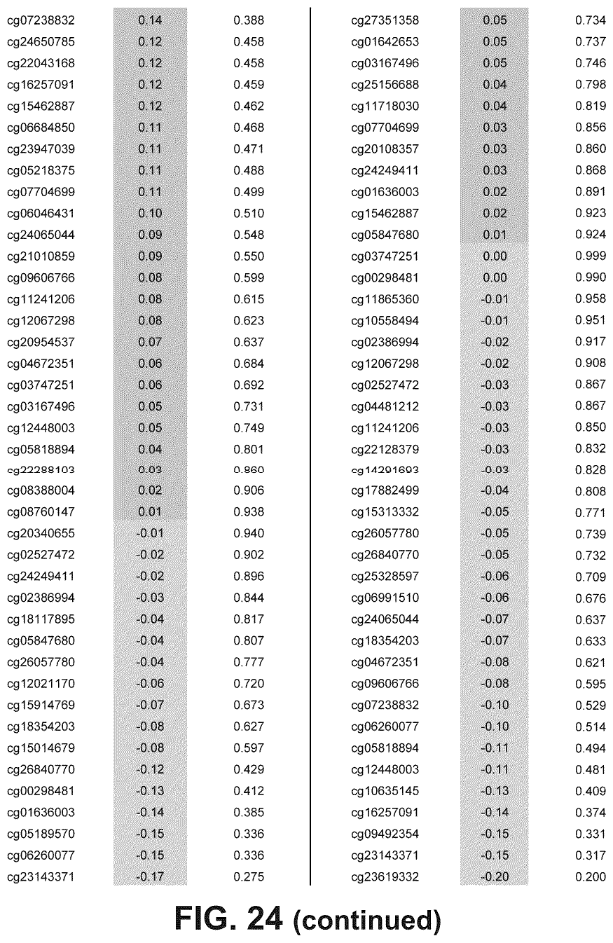

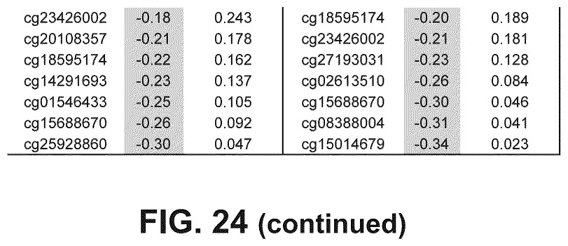

We next tested DNAm differences directly between delirium cases and non-delirium controls from our ongoing study. Whole blood samples from 43 delirium and 44 non-delirium inpatient subjects (age average 70.2 years, SD 10.2, range 42-101 years) were analyzed using the EPIC array for genome-wide DNAm analysis. Among them, we specifically tested correlation between age and DNAm levels at CpGs in the BDNF (FIG. 24), GDNF (FIG. 21), and NR4A2 (FIG. 23) genes. There were more nominal significant (p<0.05) CpGs in delirium cases than in non-delirium controls with BDNF, GDNF, and NR4A2. Also, there were more positively correlated CpGs in delirium cases than in non-delirium controls with BDNF, GDNF, and NPAS4. Furthermore, delirium cases showed 1 significant CpG after correction for multiple testing level (p<0.05/192=7.35×E−4) in BDNF (FIG. 24), whereas non-delirium controls showed no significant CpGs after correction for multiple testing level.

### DISCUSSION

In the present study, data from GTP cohort blood samples confirmed that the DNAm patterns showed mostly positive correlation with aging among nominal significant CpG sites in neurotrophic genes. Also, among the multiple significant testing levels, almost all of the significant CpGs were positively correlated with aging. These results showed that DNAm level of neurotrophic factors correlate with aging, possibly suggesting that expression of neurotrophic factors decreases along with aging, leading to impaired cognition. Because expression data in blood from this cohort did not show age associated decrease, further research is needed to confirm this possibility, especially using brain samples.

Data from NSG cohort brain samples also confirmed that almost all the DNAm patterns showed positive correlation with aging among nominally significant neurotrophic genes (BDNF, GDNF, NR4A2, and NPAS4). Thus, we were able to confirm the same tendency for DNAm among neurotrophic genes to increase with aging both in the GTP blood samples and the NSG brain samples. Furthermore, the top hit CpG in the GTP study with blood samples was also positively correlated with aging at multiple significant testing levels in the NSG study with brain samples. These results indicated that DNAm pattern in neurotrophic genes were the same both in brain and blood tissues, and were consistent with our hypothesis.

Data from EOD cohort blood samples confirmed that DNAm levels among neurotrophic genes (BDNF, GDNF, NR4A2, and NPAS4) were positively correlated with aging in delirium cases rather than non-delirium controls. This is consistent with our hypothesis that a high level of DNAm of neurotrophic genes (BDNF, GDNF, NR4A2, and NPAS4) among elderly patients can lead to decreased expression leading to delirium, whereas in controls when DNAm levels remain low and thus neurotrophic factor could remain high, elderly patients can be protected against delirium.

In the present study, we showed significant correlation between DNAm of BDNF and aging in three independent cohorts, including patients with delirium. Although the present study did not directly show data related to expression change, it is possible that increased DNAm of BDNF may be at least partially responsible for altering expression level of BDNF. Our results may suggest that DNAm can be a better method to capture subtle change associated with aging and delirium risk. There may be the same pathway through epigenetic mechanism between dementia and delirium in regards with age associated decrease in BDNF.

In previous study, we showed that DNAm level of the TNF-alpha gene was negatively correlated with aging at almost all of the CpGs, and TNF-alpha expression level was positively correlated with aging in GTP cohort. This result of DNAm level of TNF-alpha was also shown in the NSG cohort. Furthermore, DNAm level of TNF-alpha was negatively correlated with aging at multiple test adjusted significant levels only in delirium cases (under review). Thus, neuroinflammation genes, especially TNF-alpha, may play an important role in pathophysiology of delirium. In this study, we showed more associations between DNAm of neurotrophic genes and aging in delirium cases than in controls. Taken together, we speculate that DNAm of neurotrophic and neuroinflammation processes may interact together and both play important roles in development of delirium among elderly patients.

In conclusion, to the best of our knowledge, this is the first study to report the correlation between DNAm of neurotrophic genes and aging in delirium inpatients. Despite these limitations mentioned above, we showed evidence of epigenetic differences of BDNF gene between delirium cases and non-delirium controls. This finding indicates that DNAm status of BDNF gene in blood may become a useful epigenetic biomarker for delirium.

### REFERENCES

Aryee, Martin J, Jaffe, Andrew E, Corrada-Bravo, Hector, Ladd-Acosta, Christine, Feinberg, Andrew P. Hansen, Kasper D, & Irizarry, Rafael A. (2014). Minfi: a flexible and comprehensive Bioconductor package for the analysis of Infinium DNA methylation microarrays. Bioinformatics, 30(10), 1363-1369.
Assenov, Yassen, Müller, Fabian, Lutsik, Pavlo, Walter, Jörn, Lengauer, Thomas, & Bock, Christoph. (2014). Comprehensive analysis of DNA methylation data with RnBeads. Nature methods, 11 (11), 1138.
Berchtold, N. C., Cribbs, D. H., Coleman, P. D., Rogers, J., Head, E., Kim, R., . . . . Cotman, C. W. (2008). Gene expression changes in the course of normal brain aging are sexually dimorphic. Proc Natl Acad Sci USA, 105 (40), 15605-15610. doi: 10.1073/pnas.0806883105
Beveridge, N. J., Santarelli, D. M., Wang, X., Tooney, P. A., Webster, M. J., Weickert, C. S., & Cairns, M. J. (2014). Maturation of the human dorsolateral prefrontal cortex coincides with a dynamic shift in microRNA expression. Schizophr Bull, 40 (2), 399-409. doi: 10.1093/schbul/sbs198
Braun, Patricia R, Han, Shizhong, Hing, Benjamin, Nagahama, Yasunori, Gaul, Lindsey N, Heinzman, Jonathan T, . . . Howard, Matthew A. (2019). Genome-wide DNA methylation comparison between live human brain and peripheral tissues within individuals. Translational psychiatry, 9(1), 47.
Brum, C., Stertz, L., Borba, E., Rumi, D., Kapczinski, F., & Camozzato, A. (2015). Association of serum brain-derived neurotrophic factor (BDNF) and tumor necrosis factor-alpha (TNF-alpha) with diagnosis of delirium in oncology inpatients. Braz J Psychiatry, 37(3), 197-202. doi: 10.1590/1516-4446-2014-1450
Choy, Fong Chan, Klaric, Thomas S, Leong, Wai Khay, Koblar, Simon A, & Lewis, Martin D. (2016). Reduction of the neuroprotective transcription factor Npas4 results in increased neuronal necrosis, inflammation and brain lesion size following ischaemia. Journal of Cerebral Blood Flow & Metabolism, 36(8), 1449-1463.
Christensen, B. C., Houseman, E. A., Marsit, C. J., Zheng, S., Wrensch, M. R., Wiemels, J. L., . . . . Kelsey, K. T. (2009). Aging and environmental exposures alter tissue-specific DNA methylation dependent upon CpG island context. PLOS Genet, 5 (8), e1000602. doi: 10.1371/journal.pgen.1000602
Danka Mohammed, C. P., Park, J. S., Nam, H. G., & Kim, K. (2017). MicroRNAs in brain aging. Mech Ageing Dev. doi: 10.1016/j.mad.2017.01.007
Day, K., Waite, L. L., Thalacker-Mercer, A., West, A., Bamman, M. M., Brooks, J. D., . . . . Absher, D. (2013). Differential DNA methylation with age displays both common and dynamic features across human tissues that are influenced by CpG landscape. Genome Biol, 14(9), R102. doi: 10.1186/gb-2013-14-9-r102
Elie, Michel, Cole, Martin G, Primeau, François J, & Bellavance, François. (1998). Delirium risk factors in elderly hospitalized patients. Journal of general internal medicine, 13(3), 204-212.
Ely, E. W., Inouye, S. K., Bernard, G. R., Gordon, S., Francis, J., May, L., . . . . Dittus, R. (2001). Delirium in mechanically ventilated patients: validity and reliability of the confusion assessment method for the intensive care unit (CAM-ICU). JAMA, 286(21), 2703-2710.
Ely, E. W., Margolin, R., Francis, J., May, L., Truman, B., Dittus, R., . . . . Inouye, S. K. (2001). Evaluation of delirium in critically ill patients: validation of the Confusion Assessment Method for the Intensive Care Unit (CAM-ICU). Crit Care Med, 29(7), 1370-1379.
Fortin, Jean-Philippe, Triche Jr, Timothy J, & Hansen, Kasper D. (2016). Preprocessing, normalization and integration of the Illumina HumanMethylationEPIC array with minfi. Bioinformatics, 33(4), 558-560.
Fortin, Jean-Philippe, Triche, Timothy, & Hansen, Kasper. (2016). Preprocessing, normalization and integration of the Illumina HumanMethylationEPIC array. BioRxiv, 065490.
Ghitza, U. E., Zhai, H., Wu, P., Airavaara, M., Shaham, Y., & Lu, L. (2010). Role of BDNF and GDNF in drug reward and relapse: a review. Neurosci Biobehav Rev, 35(2), 157-171. doi: 10.1016/j.neubiorev.2009.11.009
Gong, H., Qian, H., Ertl, R., Astle, C. M., Wang, G. G., Harrison, D. E., & Xu, X. (2015). Histone modifications change with age, dietary restriction and rapamycin treatment in mouse brain. Oncotarget, 6(18), 15882-15890. doi: 10.18632/oncotarget.4137
Grandi, C., Tomasi, C. D., Fernandes, K., Stertz, L., Kapczinski, F., Quevedo, J., . . . . Ritter, C. (2011). Brain-derived neurotrophic factor and neuron-specific enolase, but not S100beta, levels are associated to the occurrence of delirium in intensive care unit patients. J Crit Care, 26(2), 133-137. doi: 10.1016/j.jcrc.2010.10.006
Gunstad, John, Benitez, Andreana, Smith, Joseph, Glickman, Ellen, Spitznagel, Mary B, Alexander, Thomas, . . . . Murray, Leigh. (2008). Serum brain-derived neurotrophic factor is associated with cognitive function in healthy older adults. Journal of geriatric psychiatry and neurology, 21(3), 166-170.
Ihara, K., Fuchikami, M., Hashizume, M., Okada, S., Kawai, H., Obuchi, S., . . . . Morinobu, S. (2018). The influence of aging on the methylation status of brain-derived neurotrophic factor gene in blood. Int J Geriatr Psychiatry, 33(10), 1312-1318. doi: 10.1002/gps.4927
Inouye, S. K. (2006). Delirium in older persons. N Engl J Med, 354(11), 1157-1165. doi: 10.1056/NEJMra052321
Johnson, W. E., Li. C., & Rabinovic, A. (2007). Adjusting batch effects in microarray expression data using empirical Bayes methods. Biostatistics, 8(1), 118-127. doi: 10.1093/biostatistics/kxj037
Kang, H. J., Kawasawa, Y. I., Cheng, F., Zhu, Y., Xu, X., Li. M., . . . Sestan, N. (2011). Spatio-temporal transcriptome of the human brain. Nature, 478(7370), 483-489. doi: 10.1038/nature10523
Laske, Christoph, Stransky, Elke, Leyhe, Thomas, Eschweiler, Gerhard W, Maetzler, Walter, Wittorf, Andreas, . . . . Bartels, Mathias. (2007). BDNF serum and CSF concentrations in Alzheimer's disease, normal pressure hydrocephalus and healthy controls. Journal of psychiatric research, 41(5), 387-394.
Leek, Jeffrey T., Johnson, W. Evan, Parker, Hilary S., Fertig, Elana J., Jaffe, Andrew E., Storey, John D., . . . . Torres, Leonardo Collado. (2019). sva: Surrogate variable analysis. R package version.
Li, Ge, Peskind, Elaine R, Millard, Steven P. Chi, Peter, Sokal, Izabela, Yu, Chang-En, . . . . Montine, Thomas J. (2009). Cerebrospinal fluid concentration of brain-derived neurotrophic factor and cognitive function in non-demented subjects. PloS one, 4(5), e5424.
Mei, X., & Tong, J. (2016). The plasma levels of brain-derived neurotrophic factor are positively associated with emergence agitation in the elderly after gastrointestinal surgery. J Anesth, 30(5), 811-816. doi: 10.1007/s00540-016-2212-3
Moon, Minho, Jung, Eun Sun, Jeon, Seong Gak, Cha, Moon-Yong, Jang, Yongwoo, Kim, Woori, . . . . Kim, Kwang-Soo. (2019). Nurr1 (NR4A2) regulates Alzheimer's disease-related pathogenesis and cognitive function in the 5×FAD mouse model. Aging cell, 18(1), e12866.
Morris, T. J., Butcher, L. M., Feber, A., Teschendorff. A. E., Chakravarthy, A. R., Wojdacz, T. K., & Beck, S. (2014). ChAMP: 450k Chip Analysis Methylation Pipeline. Bioinformatics, 30(3), 428-430. doi: 10.1093/bioinformatics/btt684
Nishimura, K., Yokoyama, K., Yamauchi, N., Koizumi, M., Harasawa, N., Yasuda, T . . . investigators, Tmad. (2016). Sensitivity and specificity of the Confusion Assessment Method for the Intensive Care Unit (CAM-ICU) and the Intensive Care Delirium Screening Checklist (ICDSC) for detecting post-cardiac surgery delirium: A single-center study in Japan. Heart Lung, 45(1), 15-20. doi: 10.1016/j.hrtlng.2015.11.001
Nordlund, Jessica, Bäcklin, Christofer L, Wahlberg, Per, Busche, Stephan, Berglund, Eva C, Eloranta, Maija-Leena, . . . . Harila-Saari, Arja. (2013). Genome-wide signatures of differential DNA methylation in pediatric acute lymphoblastic leukemia. Genome biology, 14 (9), r105.
Numata, S., Ye, T., Hyde, T. M., Guitart-Navarro, X., Tao, R., Wininger, M., . . . . Lipska, B. K. (2012). DNA methylation signatures in development and aging of the human prefrontal cortex. Am J Hum Genet, 90(2), 260-272. doi: 10.1016/j.ajhg.2011.12.020
Pal. S., & Tyler, J. K. (2016). Epigenetics and aging. Sci Adv, 2(7), e1600584. doi: 10.1126/sciadv.1600584
Pandharipande, P. P., Girard, T. D., Jackson, J. C., Morandi, A., Thompson, J. L., Pun, B. T . . . . Ely, E. W. (2013). Long-term cognitive impairment after critical illness. N Engl J Med, 369(14), 1306-1316. doi: 10.1056/NEJMoa1301372
Phillips, Heidi S, Hains, Jeanne M, Armanini, Mark, Laramee, Gary R, Johnson, Steven A, & Winslow, John W. (1991). BDNF mRNA is decreased in the hippocampus of individuals with Alzheimer's disease. Neuron, 7(5), 695-702.
R Core Team. (2019). R: A language and environment for statistical computing. Retrieved accessed 23 Apr. 2019, from R Foundation for Statistical Computing https://www.R-project.org/
Ratanatharathorn, Andrew, Boks, Marco P, Maihofer, Adam X, Aiello, Allison E, Amstadter, Ananda B. Ashley-Koch, Allison E, . . . . Dennis, Michelle. (2017). Epigenome-wide association of PTSD from heterogeneous cohorts with a common multi-site analysis pipeline. American Journal of Medical Genetics Part B: Neuropsychiatric Genetics, 174(6), 619-630.
Rockwood, K., Cosway, S., Carver, D., Jarrett, P., Stadnyk, K., & Fisk, J. (1999). The risk of dementia and death after delirium. Age Ageing, 28(6), 551-556. doi: 10.1093/ageing/28.6.551
Schuurmans, M. J., Shortridge-Baggett, L. M., & Duursma, S. A. (2003). The Delirium Observation Screening Scale: a screening instrument for delirium. Res Theory Nurs Pract, 17(1), 31-50.
Sen, P., Shah, P. P., Nativio, R., & Berger, S. L. (2016). Epigenetic Mechanisms of Longevity and Aging. Cell, 166(4), 822-839. doi: 10.1016/j.cell.2016.07.050
Shimada, Hiroyuki, Makizako, Hyuma, Yoshida, Daisuke, Tsutsumimoto, Kota, Anan, Yuya, Uemura, Kazuki, . . . . Suzuki, Takao. (2014). A large, cross-sectional observational study of serum BDNF, cognitive function, and mild cognitive impairment in the elderly. Frontiers in aging neuroscience, 6, 69.
Shinozaki, G., Bormann, N. L., Chan, A. C., Zarei, K., Sparr, N. A., Klisares, M. J . . . . Cromwell, J. (2019). Identification of high mortality risk patients and prediction of outcomes in delirium by Bispectral EEG. Journal of Clinical Psychiatry, in press.
Shinozaki, G., Braun, P. R., Hing, B. W. Q., Ratanatharathorn, A., Klisares, M. J., Duncan, G. N . . . . Cho, H. R. (2018). Epigenetics of Delirium and Aging: Potential Role of DNA Methylation Change on Cytokine Genes in Glia and Blood Along With Aging. Front Aging Neurosci, 10, 311. doi: 10.3389/fnagi.2018.00311
Shinozaki, G., Chan, A. C., Sparr, N. A., Zarei, K., Gaul, L. N., Heinzman, J. T., . . . . Cromwell, J. W. (2018). Delirium detection by a novel bispectral electroencephalography device in general hospital. Psychiatry Clin Neurosci, 72(12), 856-863. doi: 10.1111/pcn.12783
Spronk, P. E., Riekerk, B., Hofhuis, J., & Rommes, J. H. (2009). Occurrence of delirium is severely underestimated in the ICU during daily care. Intensive Care Med, 35(7), 1276-1280. doi: 10.1007/s00134-009-1466-8
Teschendorff, A. E., Marabita, F., Lechner, M., Bartlett, T., Tegner, J., Gomez-Cabrero, D., & Beck, S. (2013). A beta-mixture quantile normalization method for correcting probe design bias in Illumina Infinium 450 k DNA methylation data. Bioinformatics, 29(2), 189-196. doi: 10.1093/bioinformatics/bts680
Trzepacz, P. T., Mittal, D., Torres, R., Kanary, K., Norton, J., & Jimerson, N. (2001). Validation of the Delirium Rating Scale-revised-98: comparison with the delirium rating scale and the cognitive test for delirium. J Neuropsychiatry Clin Neurosci, 13(2), 229-242. doi: 10.1176/jnp.13.2.229
Tsai, P. C., Spector, T. D., & Bell, J. T. (2012). Using epigenome-wide association scans of DNA methylation in age-related complex human traits. Epigenomics, 4(5), 511-526. doi: 10.2217/epi. 12.45
van Eijk, M. M., van den Boogaard, M., van Marum, R. J., Benner, P., Eikelenboom, P., Honing, M. L., . . . . Slooter, A. J. (2011). Routine use of the confusion assessment method for the intensive care unit: a multicenter study. Am J Respir Crit Care Med, 184(3), 340-344. doi: 10.1164/rccm.201101-00650C
Williams, J., Finn, K., Melvin, V., Meagher, D., McCarthy, G., & Adamis, D. (2017). The Association of Serum Levels of Brain-Derived Neurotrophic Factor with the Occurrence of and Recovery from Delirium in Older Medical Inpatients. Biomed Res Int, 2017, 5271395. doi: 10.1155/2017/5271395
Wyrobek, J., LaFlam, A., Max, L., Tian, J., Neufeld, K. J., Kebaish, K. M., . . . . Brown, C. H. th. (2017). Association of intraoperative changes in brain-derived neurotrophic factor and postoperative delirium in older adults. Br J Anaesth, 119(2), 324-332. doi: 10.1093/bja/aex103
Zampieri, M., Ciccarone, F., Calabrese, R., Franceschi, C., Burkle, A., & Caiafa, P. (2015). Reconfiguration of DNA methylation in aging. Mech Ageing Dev, 151, 60-70. doi: 10.1016/j.mad.2015.02.002
Zhou, Wanding, Laird, Peter W, & Shen, Hui. (2017). Comprehensive characterization, annotation and innovative use of Infinium DNA methylation BeadChip probes. Nucleic acids research, 45(4), e22-e22.

### Figure Captions and Notes

FIGS. 20A and 20B: Correlation between age and beta value in the top 2 CpGs. Abbreviations: BDNF: Brain-derived neurotrophic factor, GDNF; glial cell-derived neurotrophic factor.

Table 1 (FIG. 19): Correlations between age and DNAm levels of neurotrophic genes in blood samples obtained from the GTP cohort. Notes: Blue highlights a positive correlation, and pink highlights a negative correlation. **Significant after Bonferroni corrected genome-wide significance levels (p<5×E−8), *Significant after correction for multiple testing level (p<0.05/226). Abbreviations: AP-1 Transcription Factor Subunit, ARC; activity regulated cytoskeleton associated protein, BDNF; Brain-derived neurotrophic factor, FOS; Fos Proto-Oncogene, GDNF; glial cell-derived neurotrophic factor, GTP; Grady Trauma Project, NPAS4; neuronal Per-Arnt-Sim domain protein 4, NR4A1; nuclear receptor subfamily 4A1, NR4A2; nuclear receptor subfamily 4A2.

Table 2 (FIG. 22): Correlations between age and DNAm levels of neurotrophic genes in brain samples obtained from the NSG cohort. Notes: Blue highlights a positive correlation, and pink highlights a negative correlation. *Significant after correction for multiple testing level (p<0.05/201), ^{†}nominally significant (p<0.05). Abbreviations: BDNF; Brain-derived neurotrophic factor, GDNF: glial cell-derived neurotrophic factor, NPAS4; neuronal Per-Arnt-Sim domain protein 4, NR4A2; nuclear receptor subfamily 4A2, NSG; neurosurgery.

Table 3 (FIG. 24): Correlation of age and blood DNAm at 83 CpGs in the BDNF gene compared between delirium cases vs non-delirium controls in the EOD cohort. Notes: Blue highlights a positive correlation, and pink highlights a negative correlation. *Significant after correction for multiple testing level (p<0.05/192), ^{†}nominally significant (p<0.05). Abbreviations: BDNF; Brain-derived neurotrophic factor, DNAm; DNA methylation, EOD; Epigenetics of Delirium.

Supplementary Table 1 (FIG. 21): Correlations between age and DNAm levels at 226 CpGs in neurotrophic genes in blood samples obtained from the GTP cohort. Notes: Blue highlights a positive correlation, and pink highlights a negative correlation. **Significant after Bonferroni corrected genome-wide significance levels (p<5×E−8), *Significant after correction for multiple testing level (p<0.05/226). Abbreviations: AP-1 Transcription Factor Subunit, ARC; activity regulated cytoskeleton associated protein, BDNF; Brain-derived neurotrophic factor, FOS; Fos Proto-Oncogene, GDNF; glial cell-derived neurotrophic factor, GTP; Grady Trauma Project, NPAS4; neuronal Per-Arnt-Sim domain protein 4, NR4A1; nuclear receptor subfamily 4A1, NR4A2; nuclear receptor subfamily 4A2.

Supplementary Table 2 (FIG. 23): Correlations between age and DNAm levels at 201 CpGs in neurotrophic genes in brain samples obtained from the NSG cohort. Notes: Blue highlights a positive correlation, and pink highlights a negative correlation. *Significant after correction for multiple testing level (p<0.05/201), †nominally significant (p<0.05). Abbreviations: BDNF; Brain-derived neurotrophic factor, GDNF; glial cell-derived neurotrophic factor, NPAS4; neuronal Per-Arnt-Sim domain protein 4, NR4A2; nuclear receptor subfamily 4A2, NSG; neurosurgery.

Although the disclosure has been described with reference to preferred embodiments, persons skilled in the art will recognize that changes may be made in form and detail without departing from the spirit and scope of the disclosed apparatus, systems and methods.

What is claimed is:

1. A method of determining a subject's susceptibility to delirium, comprising the steps of:
a. collecting a biological sample from the subject;
b determining the methylation status of at least one CpG sequence in the biological sample from the subject; and
c. comparing the methylation status in the biological sample to an established threshold determining whether or not the subject is likely to suffer delirium.

2. The method of claim 1, wherein the at least one CpG sequence comprises a position in a gene selected from TNF-alpha, BNDF, GDNF, LDLRAD4, DAPK1, and IRF8.

3. The method of claim 2, wherein the at least one CpG sequence comprises position 05733135 of chromosome 11 within the BDNF gene and wherein the detection of methylation indicates the subject is susceptible to delirium.

4. The method of claim 2, wherein the at least one CpG sequence comprises cg02328239 of chromosome 5 within the GDNF gene and wherein the detection of methylation indicates likelihood of experiencing delirium.

5. The method of claim 2, wherein the at least one CpG sequence selected from cg26729380, cg10650821, and cg04425624 of chromosome 6 within the TNF-alpha gene and wherein the detection of demethylation indicates likelihood of experiencing delirium.

6. The method of claim 2, wherein the at least one CpG sequence comprises cg21295729 of chromosome 18 within the LDLRAD4 gene and wherein the detection of methylation indicates likelihood of experiencing delirium.

7. The method of claim 2, wherein the at least one CpG sequence comprises cg10518911 of chromosome 9 within the DAPK1 gene and wherein the detection of methylation indicates likelihood of experiencing delirium.

8. The method of claim 2, wherein the at least one CpG sequence comprises cg4015794 of chromosome 16 within the IRF8 gene and wherein the detection of methylation indicates likelihood of experiencing delirium.

9. The method of claim 1, further comprising determining the ratio of methylated one or more CpG sequences to unmethylated one or more CpG sequences.

10. A kit for determining a subject's susceptibility to delirium by determining the methylation status of at least one CpG sequence of the subject, the kit comprising: at least one first nucleic acid primer at least 8 nucleotides in length that is complementary to a bisulfite-converted nucleic acid sequence comprising a CpG sequence at cg05733135 of chromosome 11 within the BDNF gene, wherein the at least one first nucleic acid primer detects the methylated CpG sequence.

11. The kit of claim 10, further comprising at least one second nucleic acid primer at least 8 nucleotides in length that is complementary to a bisulfite-converted nucleic acid sequence comprising a CpG sequence at cg05733135 of chromosome 11 within the BDNF gene, wherein the at least one second nucleic acid primer detects the unmethylated CpG sequence.

12. The kit of claim 10, wherein the at least one first nucleic acid primer comprises one or more synthetic or non-natural nucleotides.

13. The kit of claim 10, further comprising a solid substrate to which the at least one first nucleic acid primer is bound.

14. The kit of claim 13, wherein the substrate is a polymer, glass, semiconductor, paper, metal, gel or hydrogel.

15. The kit of claim 13, wherein the solid substrate is a microarray or microfluidics card.

16. The kit of claim 10, further comprising a detectable label.

17. The kit of claim 10, further comprising at least one second nucleic acid primer at least 8 nucleotides in length that is complementary to a bisulfite-converted nucleic acid sequence comprising a CpG sequence at cg21295729 of chromosome 18 within the LDLRAD4 gene and wherein the detection of methylation indicates increased susceptibility to delirium.

18. The kit of claim 10, further comprising at least one second nucleic acid primer at least 8 nucleotides in length that is complementary to a bisulfite-converted nucleic acid sequence comprising a CpG sequence selected from cg26729380, cg10650821, and cg4425624 of chromosome 6 within the TNF-alpha gene and wherein the detection of methylation indicates decreased susceptibility to delirium.

19. The kit of claim 10, further comprising at least one second nucleic acid primer at least 8 nucleotides in length that is complementary to a bisulfite-converted nucleic acid sequence comprising a CpG sequence at cg10518911 of chromosome 9 within the DAPK1 gene and wherein the detection of methylation indicates increased susceptibility to delirium.

20. A computer implemented method for determining whether or not a subject will experience delirium, the method comprising:
a. obtaining self-report data for a user;
b performing one or more predictive calculations to determine a predicted multisite CpG sequence methylation ratio, a predicted threshold methylation status and predicted delirium of the user;
c. providing a measured multisite CpG sequence unmethylated level and a measured multisite CpG sequence methylation status for the user;
d. generating a predictive score based on the self-report data, the one or more predictive calculations, the measured multisite CpG sequence methylation and unmethylated level and the measured multisite CpG sequence methylation ratio; and
e. outputting a predicted level of delirium based on the predictive score.
